(19) United States
(12) Patent Application Publication
(10) Pub. No.: US 2004/0097573 A1
(43) Pub. Date: May 20, 2004

(54) Use of a COX-2 inhibitor and a NK-1 receptor antagonist for treating inflammation

(76) Inventors: Susan Boyce, Bishop Stortford (GB); Raymond George Hill, Royston (GB); Nadia Melanie Rupniak, Bishop Stortford (GB)

Correspondence Address: MERCK AND CO INC, P O BOX 2000, RAHWAY, NJ, 070650907

(21) Appl. No.: 10/614,389

(22) Filed: Jul. 7, 2003

Related U.S. Application Data
(63) Continuation of application No. 09/700,776, filed on Nov. 20, 2000, now abandoned.
Application No. 09/700,776 is a 371 of international application No. PCT/GB99/01632, filed on May 19, 1999.

(30) Foreign Application Priority Data
May 21, 1998 (GB) 9810920.0

(51) Int. Cl. A61K 31/415; A61K 31/365

(52) U.S. Cl. 514/406; 514/473

(57) ABSTRACT

The present invention provides the use of a COX-2 inhibitior and an NK-1 receptor antagonist for the treatment or prevention of inflammatory disorders.

The present invention involves a drug combination comprising an inhibitor of cyclooxygenase-2 in combination with a neurokinin-1 (NK-1) receptor antagonist.

Inhibitors of cyclooxygenase-2 are a sub-class of the class of drugs known as non-steroidal antiinflammatory drugs (NSAIDs). The NSAIDs are active in reducing the prostaglandin-induced pain and swelling associated with the inflammation process but are also active in affecting other prostaglandin-regulated processes such as maintenance of the gastric lining. Thus, use of high doses of most common NSAIDs can produce severe side effects, including life threatenting ulcers, that limit their therapeutic potential. An alternative to NSAIDs is the use of corticosteroids, which have even more serious side effects, especially when long term, therapy is involved.

Previous NSAIDs have been found to prevent the production of prostaglandin by inhibiting enzymes in the human arachidonic acid/prostaglandin pathway including the enzyme cyclooxygenase (COX). There are two isoforms of the COX enzyme, the first, COX-1, is constitutively expressed and is involved with physiological functions and the second, COX-2, is induced locally in inflamed tissue. While conventional NSAIDs block both forms of the enzyme, the inducible COX-2 enzyme associated inflammation has provided a more focussed drug target which should enable effective antiinflammatory analgesia with reduced gastrointestinal side effects. Many compounds which have activity as COX-2 inhibitors have been identified, and clinical trials are reported to be in progress.

Neurokinin 1 (NK-1; substance P) receptor antagonists are being developed for the treatment of a number of physiological disorders associated with an excess or imbalance of tachykinins, and in particular substance P. Substance P has been implicated in the pathology of a number of inflammatory conditions (see, for instance, International (PCT) patent specification Nos. WO 95/16679, WO 95/18124 and WO 95/23798).

Improved therapies for treating and preventing inflammatory disorders are currently being sought for the large number of individuals who are at risk from these disorders. The present invention addresses this problem by providing a combination therapy comprised of a COX-2 inhibitor with a NK-1 receptor antagonist. When administered as part of a combination therapy, the COX-2 inhibitor together with the NK-1 receptor antagonist provide enhanced treatment options as compared with administration of either the COX-2 inhibitor or the NK-1 receptor antagonist alone.

The present invention provides a novel drug combination comprised of a COX-2 inhibitor in combination with a NK-1 receptor antagonist, which is useful for treating, preventing, reducing the progression, and/or reducing the risk of developing inflammatory disorders.

The present invention accordingly provides the use of a COX-2 inhibitor in combination with a NK-1 receptor antagonist for the manufacture of a medicament for the treatment or prevention of inflammatory disorders.

The present invention also provides a method for the treatment or prevention of inflammatory disorders, which method comprises administration to a patient in need of such treatment an amount of a COX-2 inhibitor and an amount of a NK-1 receptor antagonist, such that together they give effective relief.

In a further aspect of the present invention, there is provided a pharmaceutical composition for the treatment or prevention of inflammatory disorders comprising a COX-2 inhibitor and a NK-1 receptor antagonist, together with at least one pharmaceutically acceptable carrier or excipient.

It will be appreciated that the COX-2 inhibitor and the NK-1 receptor antagonist, may be present as a combined preparation for simultaneous, separate or sequential use for the treatment or prevention of inflammatory disorders. Such combined preparations may be, for example, in the form of a twin pack.

In a further or alternative aspect of the present invention, there is therefore provided a product comprising a COX-2 inhibitor and a NK-1 receptor antagonist as a combined preparation for simultaneous, separate or sequential use in the treatment or prevention of inflammatory disorders.

It will be appreciated that when using a combination of the present invention, both the COX-2 inhibitor and the NK-1 receptor antagonist will be administered to a patient, within a reasonable period of time. The compounds may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially. Therefore, by way of example, the COX-2 inhibitor may be administered as a tablet and then, within a reasonable period of time, the NK-1 receptor antagonist may be administered either as an oral dosage form such as a tablet or a fast-dissolving oral dosage form. By a “fast dissolving oral formulation” is meant, an oral delivery form which when placed on the tongue of a patient, dissolves within about 10 seconds.

By “reasonable period of time” is meant a time period that is not in excess of about 1 hour. That is, for example, if the COX-2 inhibitor is provided as a tablet, then within one hour, the NK-1 receptor antagonist should be administered, either in the same type of dosage form, or another dosage form which provides effective delivery of the medicament.

It will be appreciated that the combination of the present invention will be particularly useful in the treatment of a COX-2 mediated disease or disorder. COX-2 mediated diseases and disorders includes inflammatory diseases susceptible to treatment with a non-steroidal anti-inflammatory agent. Such “inflammatory disorders” include rheumatoid arthritis, degenerative Joint diseases (osteoarthritis), bursitis, tendinitis, ankylosing spondylitis, gout and synovitis.

The terms “inhibitor of cyclooxygenase-2”, “cyclooxygenase-2 inhibitor” and “COX-2 inhibitor” as used herein embrace compounds which selectively inhibit cyclooxygenase-2 over cyclooxygenase-1. Employing the human whole blood COX-1 assay and the human whole blood COX-2 assay described in C. Brideau et al, Inflamm. Res. 45: 68-74 (1996), herein incorporated by reference, preferably, the compounds have a cyclooxygenase-2 IC₅₀ of less than about 2 μM in the human whole blood COX-2 assay, yet have a cyclooxygenase-1 IC₅₀ of greater than about 5 μM in the human whole blood COX-1 assay. Also preferably, the compounds have a selectivity ratio of cyclooxygenase-2 inhibition over cyclooxygenase-1 inhibition of at least 10, and more preferably of at least 40. The resulting selectivity may indicate an ability to reduce the incidence of common NSAID-induced side effects.

As explained in J. Talley, Exp. Opin. Ther. Patents (1997), 7(1), pp. 55-62, three distinct structural classes of selective COX-2 inhibitor compounds have been identified. One class is the methane sulfonanilide class of inhibitors, of which NS-398, flosulide, nimesulide and L-745,337 are example members.

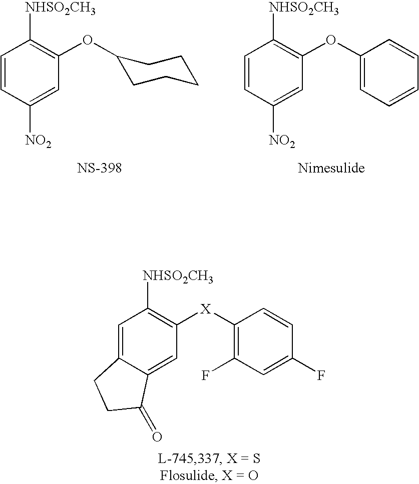

A second class is the tricyclic inhibitor class, which can be further divided into the sub-classes of tricyclic inhibitors with a central carbocyclic ring (examples include SC-57666, 1, and 2); those with a central monocyclic heterocyclic ring (examples include DuP 697, SC-58125, SC-58635, and 3, 4 and 5); and those with a central bicyclic heterocyclic ring (examples include 6, 7, 8, 9 and 10). Compounds 3, 4 and 5 are described in U.S. Pat. No. 5,474,995.

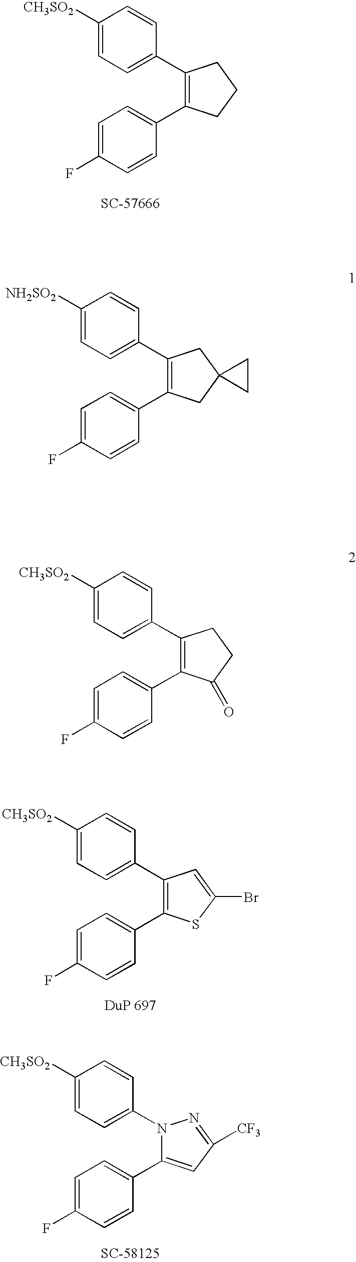

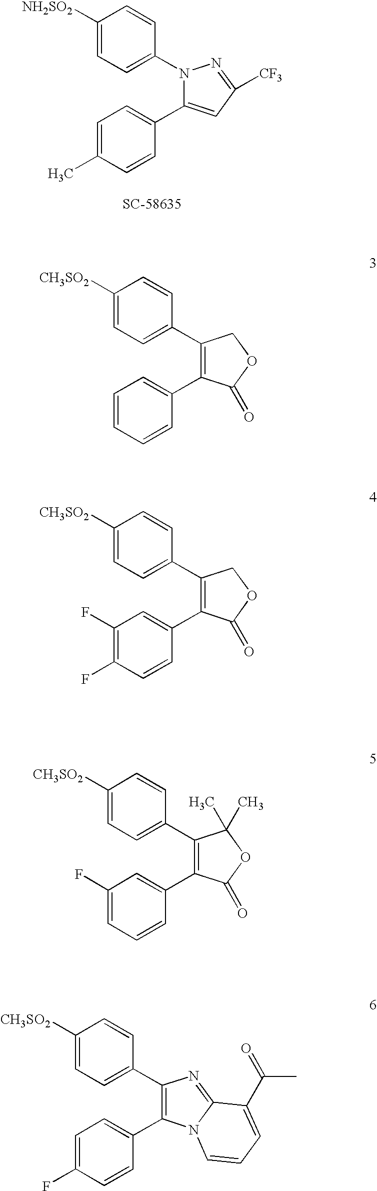

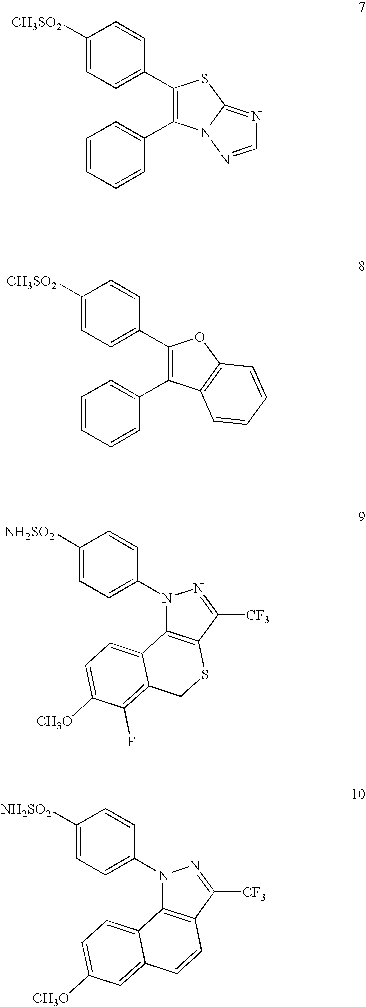

The third identified class can be referred to as those which are structurally modified NSAIDS, and includes L-761,066 and structure 11 as example members.

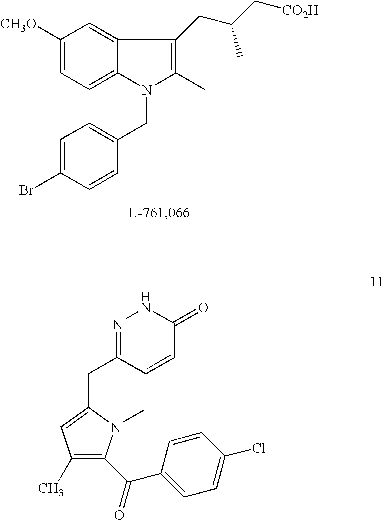

In addition to the structural classes, sub-classes, specific COX-2 inhibitor compound examples, and reference journal and patent publications described in the Talley publication which are all herein incorporated by reference, examples of compounds which selectively inhibit cyclooxygenase-2 have also been described in the following patent publications, all of which are herein incorporated by reference: U.S. Pat. Nos. 5,344,991, 5,380,738, 5,393,790, 5,409,944, 5,434,178, 5,436,265, 5,466,823, 5,474,995, 5,510,368, 5,536,752, 5,550,142, 5,552,422, 5,604,253, 5,604,260, 5,639,780; and International Patent Specification Nos. 94/13635, 94/15932, 94/20480, 94/26731, 94/27980, 95/00501, 95/15316, 96/03387, 96/03388, 96/06840; and International Publication No.'s WO 94/20480, WO 96/21667, WO 96/31509, WO 96/36623, WO 97/14691, WO 97/16435.

Additional COX-2 inhibitor compounds which are included in the scope of this invention include:

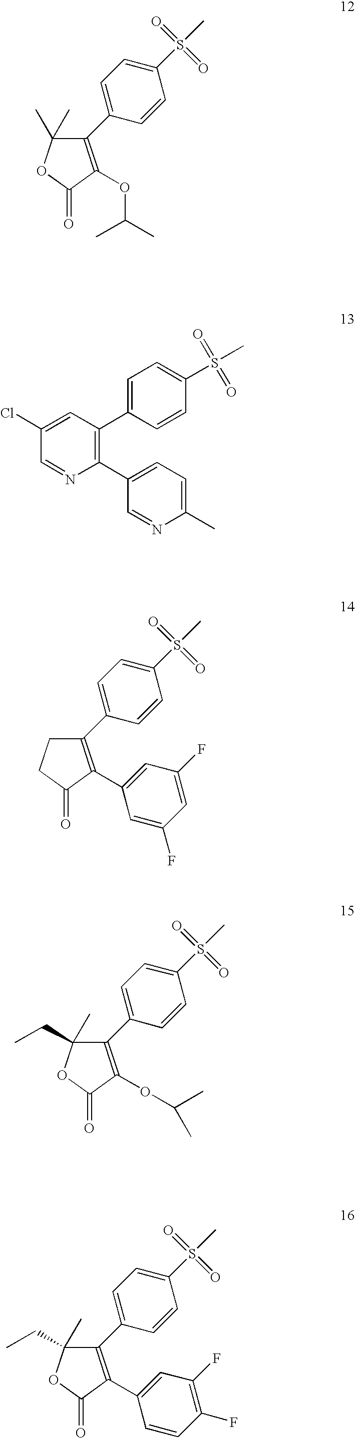

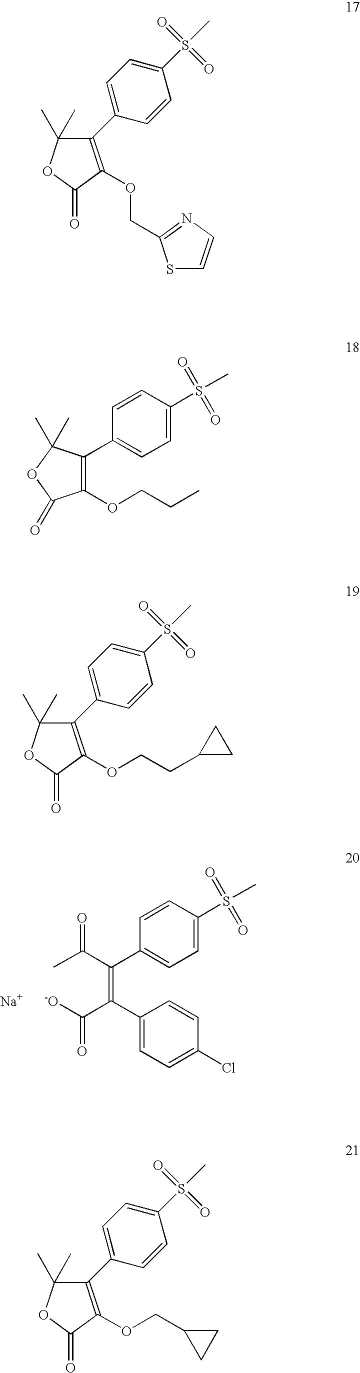

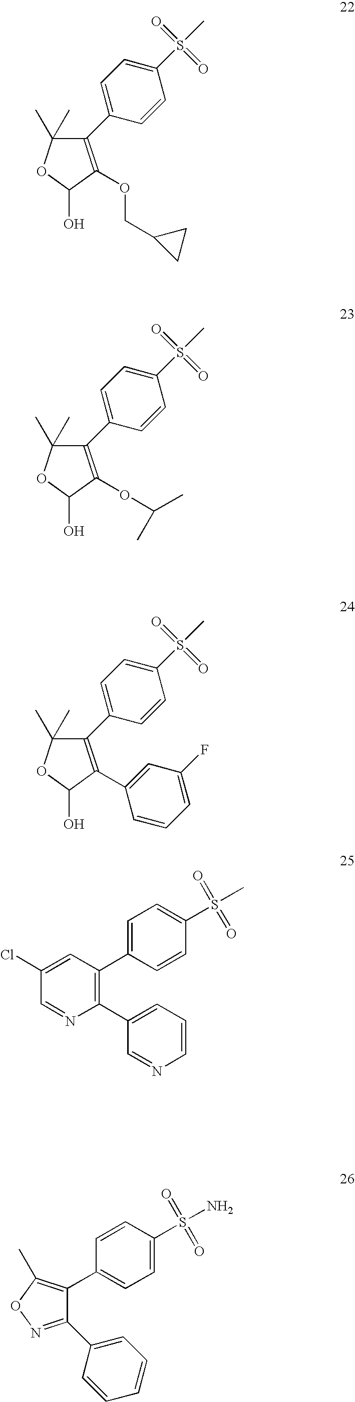

Some of the compounds above can also be identified by the following chemical names:

3: 3-phenyl-4-(4-(methylsulfonyl)phenyl)-2-(5H)-furanone;

4: 3-(3,4-difluorophenyl)-4-(4-(methylsulfonyl)phenyl)-2-(5H)-furanone;

5: 5,5-dimethyl-4-(4-(methylsulfonyl)phenyl)-3-(3-fluorophenyl)-5H-furan-2-one;

12: 5,5-dimethyl-4-(4-(methylsulfonyl)phenyl)-3-(2-propoxy)-5H-furan-2-one;

13: 5-chloro-3-(4-(methylsulfonyl)phenyl)-2-(2-methyl-5-pyridinyl)pyridine;

14: 2-(3,5-difluorophenyl)-3-(4-(methylsulfonyl)phenyl)-2-cyclopenten-1-one;

15: 5(S)-5-ethyl-5-methyl-4-(4-(methylsulfonyl)phenyl)-3-(2-propoxy)-5H-furan-2-one;

16: 5-ethyl-5-methyl-4-(4-(methylsulfonyl)phenyl)-3-(3,4-difluorophenyl)-5H-furan-2-one;

17: 3-((2-thiazolyl)methoxy)-4-(4-(methylsulfonyl)phenyl)-5,5-dimethyl-5H-furan-2-one;

18: 3-propyloxy-4-(4-(methylsulfonyl)phenyl)-5,5-dimethyl-5H-furan-2-one;

19: 3-(11-cyclopropylethoxy)-5,5-dimethyl-4-(4-methylsulfonyl)phenyl)-5H-furan-2-one;

20: sodium 2-(4-chlorophenyl)-3-(4-(methylsulfonyl)phenyl)-4-oxo-2-pentenoate;

21: 3-(cyclopropylmethoxy)-5,5-dimethyl-4-(4-(methylsulfonyl)phenyl)-5H-furan-2-one;

22: 3-(cyclopropylmethoxy)-5,5-dimethyl-4-(4-(methylsulfonyl)phenyl)-2,5-dihydrofuran-2-ol;

23: 3-isopropoxy-5,5-dimethyl-4-(4-(methylsulfonyl)phenyl)-2,5-dihydrofuran-2-ol;

24: 5,5-dimethyl-3-(3-fluorophenyl)-2-hydroxy-4-(4-(methylsulfonyl)phenyl)-2,5-dihydrofuran;

25: 5-chloro-3-(4-(methylsulfonyl)phenyl)-2-(3-pyridinyl)pyridine;

26: 4-(5-methyl-3-phenyl-4-isoxazolyl)benzenesulfonamide.

The following publications describe and/or provide methods for making the compounds as indicated: compounds 12, 15, 17, 18, 19 and 21, WO 97/14691; compounds 22, 23 and 24, WO 97/16435; compound 20, WO 96/36623, compound 14, U.S. Pat. No. 5,536,752; compound 16, U.S. Pat. No. 5,474,995. See Examples herein for compounds 13 and 25; compound 26, U.S. Pat. No. 5,633,272.

Also incorporated herein by reference are those compounds described in WO 96/41645 as having structural Formula I, shown below, and the definition and preferred definitions and species described therein:

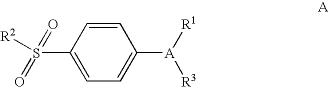

Particularly preferred compounds of formula (I) include:

5-(4-fluorophenyl)-1-[4-(methylsulfonyl)phenyl]-3-(trifluoromethyl)pyrazole;

4-(4-fluorophenyl)-5-[4-(methylsulfonyl)phenyl]-1-phenyl-3-(trifluoromethyl)pyrazole;

4-(5-(4-chlorophenyl)-3-(4-methoxyphenyl)-1H-pyrazol-1-yl)benzenesulfonamide;

4-(3,5-bis(4-methylphenyl)-1H-pyrazol-1-yl)benzenesulfonamide;

4-(5-(4-chlorophenyl)-3-phenyl-1H-pyrazol-1-yl)benzenesulfonamide;

4-(3,5-bis(4-methoxyphenyl)-1H-pyrazol-1-yl)benzenesulfonamide;

4-(5-(4-chlorophenyl)-3-(4-methylphenyl)-1H-pyrazol-1-yl)benzenesulfonamide;

4-(5-(4-chlorophenyl)-3-(4-nitrophenyl)-1H-pyrazol-1-yl)benzenesulfonamide;

4-(5-(4-chlorophenyl)-3-(5-chloro-2-thienyl)-1H-pyrazol-1-yl)benzenesulfonamide;

4-(4-chloro-3,5-diphenyl-1H-pyrazol-1-yl)benzenesulfonamide;

4-(5-(4-chlorophenyl)-3-(trifluoromethyl)-1H-pyrazol-1-yl)benzenesulfonamide;

4-(5-phenyl)-3-(trifluoromethyl)-1H-pyrazol-1-yl)benzenesulfonamide;

4-(5-(4-fluorophenyl)-3-(trifluoromethyl)-1H-pyrazol-1-yl)benzenesulfonamide;

4-(5-(4-methoxyphenyl)-3-(trifluoromethyl)-1H-pyrazol-1-yl)benzenesulfonamide;

4-(5-(4-chlorophenyl)-3-(difluoromethyl)-1H-pyrazol-1-yl)benzenesulfonamide;

4-(5-(4-methylphenyl)-3-(trifluoromethyl)-1H-pyrazol-1-yl)benzenesulfonamide;

4-(4-chloro-5-(4-chlorophenyl)-3-(trifluoromethyl)-1H-pyrazol-1-yl)benzenesulfonamide;

4-(3-(difluoromethyl)-5-(4-methylphenyl)-1H-pyrazol-1-yl)benzenesulfonamide;

4-(3-(difluoromethyl)-5-phenyl-1H-pyrazol-1-yl)benzenesulfonamide;

4-(3-(difluoromethyl)-5-(4-methoxyphenyl)-1H-pyrazol-1-yl)benzenesulfonamide;

4-(3-cyano-5-(4-fluorophenyl)-1H-pyrazol-1-yl)benzenesulfonamide;

4-(3-(difluoromethyl)-5-(3-fluoro-4-methoxyphenyl)-1H-pyrazol-1-yl)benzenesulfonamide;

4-(5-(3-fluoro-4-methoxyphenyl)-3-(trifluoromethyl)-1H-pyrazol-1-yl)benzenesulfonamide;

4-(4-chloro-5-phenyl-1H-pyrazol-1-yl)benzenesulfonamide;

4-(5-(4-chlorophenyl)-3-(hydroxyphenyl)-1H-pyrazol-1-yl)benzenesulfonamide;

4-(5-(4-(N,N-dimethylamino)phenyl)-3-(trifluoromethyl)-1H-pyrazol-1-yl)benzenesulfonamide;

5-(4-fluorophenyl)-6-(4-(methylsulfonyl)phenyl)spiro[2.4]hept-5-ene;

4-(6-(4-fluorophenyl)spiro[2.4]hept-5-en-5-yl)benzenesulfonamide;

6-(4-fluorophenyl)-7-(4-(methylsulfonyl)phenyl)spiro[3.4]oct-6-ene;

5-(3-chloro-4-methoxyphenyl)-6-(4-(methylsulfonyl)phenyl)spiro[2.4]hept-5-ene;

4-(6-(3-chloro-4-methoxyphenyl)spiro[2.4]hept-5-en-5-yl)benzenesulfonamide;

5-(3,5-dichloro-4-methoxyphenyl)-6-(4-(methylsulfonyl)phenyl)spiro[2.4]hept-5-ene;

5-(3-chloro-4-fluorophenyl)-6-(4-(methylsulfonyl)phenyl)spiro[2.4]hept-5-ene;

4-(6-(3,4-dichlorophenyl)spiro[2.4]hept-5-en-5-yl)benzenesulfonamide;

2-(3-chloro-4-fluorophenyl)-4-(4-fluorophenyl)-5-(4-methylsulfonylphenyl)thiazole;

2-(2-chlorophenyl)-4-(4-fluorophenyl)-5-(4-methylsulfonylphenyl)thiazole;

5-(4-fluorophenyl)-4-(4-methylsulfonylphenyl)-2-methylthiazole;

4-(4-fluorophenyl)-5-(4-methylsulfonylphenyl)-2-trifluoromethylthiazole;

4-(4-fluorophenyl)-5-(4-methylsulfonylphenyl)-2-(2-thienyl)thiazole;

4-(4-fluorophenyl)-5-(4-methylsulfonylphenyl)-2-benzylaminothiazole;

4-(4-fluorophenyl)-5-(4-methylsulfonylphenyl)-2-(1-propylamino)thiazole;

2-((3,5-dichlorophenoxy)methyl)-4-(4-fluorophenyl)-5-(4-(methylsulfonyl)phenyl)thiazole;

5-(4-fluorophenyl)-4-(4-methylsulfonylphenyl)-2-trifluoromethylthiazole;

1-methylsulfonyl-4-(1,1-dimethyl-4-(4-fluorophenyl)cyclopenta-2,4-dien-3-yl)benzene;

4-(4-(4-fluorophenyl)-1,1-dimethylcyclopenta-2,4-dien-3-yl)benzenesulfonamide;

5-(4-fluorophenyl)-6-(4-(methylsulfonyl)phenyl)spiro[2.4]hepta-4,6-diene;

4-(6-(4-fluorophenyl)spiro[2.4]hepta-4,6-dien-5-yl)benzenesulfonamide;

6-(4-fluorophenyl)-2-methoxy-5-(4-(methylsulfonyl)phenyl)-pyridine-3-carbonitrile;

2-bromo-6-(4-fluorophenyl)-5-(4-(methylsulfonyl)phenyl)-pyridine-3-carbonitrile;

6-(4-fluorophenyl)-5-(4-(methylsulfonyl)phenyl)-2-phenyl-pyridine-3-carbonitrile;

4-(2-(4-methylpyridin-2-yl)-4-(trifluoromethyl)-1H-imidazol-1-yl)benzenesulfonamide;

4-(2-(5-methylpyridin-3-yl)-4-(trifluoromethyl)-1H-imidazol-1-yl)benzenesulfonamide;

4-(2-(2-methylpyridin-3-yl)-4-(trifluoromethyl)-1H-imidazol-1-yl)benzenesulfonamide;

3-(1-(4-(methylsulfonyl)phenyl)-4-(trifluoromethyl)-1H-imidazol-2-yl)benzenesulfonamide;

2-(1-(4-(methylsulfonyl)phenyl)-4-(trifluoromethyl)-1H-imidazol-2-yl)pyridine;

2-methyl-4-(1-(4-(methylsulfonyl)phenyl)-4-(trifluoromethyl)-1H-imidazol-2-yl)pyridine;

2-methyl-6-(1-(4-(methylsulfonyl)phenyl)-4-(trifluoromethyl)-1H-imidazol-2-yl)pyridine;

4-(2-(6-methylpyridin-3-yl)-4-(trifluoromethyl)-1H-imidazol-1-yl)benzenesulfonamide;

2-(3,4-difluorophenyl)-1-(4-(methylsulfonyl)phenyl)-4-(trifluoromethyl)-1H-imidazole;

4-(2-(4-methylphenyl)-4-(trifluoromethyl)-1H-imidazol-1-yl)benzenesulfonamide;

2-(4-chlorophenyl)-1-(4-(methylsulfonyl)phenyl)-4-methyl-1H-imidazole;

2-(4-chlorophenyl)-1-(4-(methylsulfonyl)phenyl)-4-phenyl-1H-imidazole;

2-(4-chlorophenyl)-4-(4-fluorophenyl)-1-(4-(methylsulfonyl)phenyl)-1H-imidazole;

2-(3-fluoro-4-methoxyphenyl)-1-(4-(methylsulfonyl)phenyl)-4-(trifluoromethyl)-1H-imidazole;

1-(4-(methylsulfonyl)phenyl)-2-phenyl-4-trifluoromethyl-1H-imidazole;

2-(4-methylphenyl)-1-(4-(methylsulfonyl)phenyl)-4-trifluoromethyl-1H-imidazole;

4-(2-(3-chloro-4-methylphenyl)-4-(trifluoromethyl)-1H-imidazol-1-yl)benzenesulfonamide;

2-(3-fluoro-5-methylphenyl)-1-(4-(methylsulfonyl)phenyl)-4-(trifluoromethyl)-1H-imidazole;

4-(2-(3-fluoro-5-methylphenyl)-4-(trifluoromethyl)-1H-imidazol-1-yl)benzenesulfonamide;

2-(3-methylphenyl)-1-(4-(methylsulfonyl)phenyl)-4-(trifluoromethyl)-1H-imidazole;

4-(2-(3-methylphenyl)-4-(trifluoromethyl)-1H-imidazol-1-yl)benzenesulfonamide;

1-(4-(methylsulfonyl)phenyl)-2-(3-chlorophenyl)-4-(trifluoromethyl)-1H-imidazole;

4-(2-(3-chlorophenyl)-4-(trifluoromethyl)-1H-imidazol-1-yl)benzenesulfonamide;

4-(2-phenyl-4-(trifluoromethyl)-1H-imidazol-1-yl)benzenesulfonamide;

4-(2-(4-M ethoxy-3-chlorophenyl)-4-(trifluoromethyl)-1H-imidazol-1-yl)benzenesulfonamide;

1-allyl-4-(4-fluorophenyl)-3-(4-(methylsulfonyl)phenyl)-5-(trifluoromethyl)-1H-pyrazole;

4-(1-ethyl-4-(4-fluorophenyl)-5-(trifluoromethyl)-1H-pyrazol-3-yl)benzenesulfonamide;

N-phenyl-(4-(4-fluorophenyl)-3-(4-(methylsulfonyl)phenyl)-5-(trifluoromethyl)-1H-pyrazol-1-yl)acetamide;

ethyl(4-(4-fluorophenyl)-3-(4-(methylsulfonyl)phenyl)-5-(trifluoromethyl)-1H-pyrazol-1-yl)acetate;

4-(4-fluorophenyl)-3-(4-(methylsulfonyl)phenyl)-1-(2-phenylethyl)-1H-pyrazole;

4-(4-fluorophenyl)-3-(4-(methylsulfonyl)phenyl)-1-(2-phenylethyl)-5-(trifluoromethyl)pyrazole;

1-ethyl-4-(4-fluorophenyl)-3-(4-(methylsulfonyl)phenyl)-5-(trifluoromethyl)-1H-pyrazole;

5-(4-fluorophenyl)-4-(4-(methylsulfonyl)phenyl)-2-(trifluoromethyl)-1H-imidazole;

4-(4-(methylsulfonyl)phenyl)-5-(2-thiophenyl)-2-(trifluoromethyl)-1H-imidazole;

5-(4-fluorophenyl)-2-methoxy-4-(4-(methylsulfonyl)phenyl)-6-(trifluoromethyl)pyridine;

2-ethoxy-5-(4-fluorophenyl)-4-(4-(methylsulfonyl)phenyl)-6-(trifluoromethyl)pyridine;

5-(4-fluorophenyl)-4-(4-(methylsulfonyl)phenyl)-2-(2-propynyloxy)-6-(trifluoromethyl)pyridine;

2-bromo-5-(4-fluorophenyl)-4-(4-(methylsulfonyl)phenyl)-6-(trifluoromethyl)pyridine;

4-(2-(3-chloro-4-methoxyphenyl)-4,5-difluorophenyl)benzenesulfonamide;

1-(4-fluorophenyl)-2-(4-(methylsulfonyl)phenyl)benzene;

5-difluoromethyl-4-(4-(methylsulfonyl)phenyl)-3-phenylisoxazole;

4-(3-ethyl-5-phenylisoxazol-4-yl)benzenesulfonamide;

4-(5-difluoromethyl-3-phenylisoxazol-4-yl)benzenesulfonamide;

4-(5-hydyoxymethyl-3-phenylisoxazol-4-yl)benzenesulfonamide;

4-(5-methyl-3-phenylisoxazol-4-yl)benzenesulfonamide;

1-(2-(4-fluorophenyl)cyclopenten-1-yl)-4-(methylsulfonyl)benzene;

1-(2-(4-fluoro-2-methylphenyl)cyclopenten-1-yl)-4-(methylsulfonyl)benzene;

1-(2-(4-chlorophenyl)cyclopenten-1-yl)-4-(methylsulfonyl)benzene;

1-(2-(2,4-dichlorophenyl)cyclopenten-1-yl)-4-(methylsulfonyl)benzene;

1-(2-(4-trifluoromethylphenyl)cyclopenten-1-yl)-4-(methylsulfonyl)benzene;

1-(2-(4-methylthiophenyl)cyclopenten-1-yl)-4-(methylsulfonyl)benzene;

1-(2-(4-fluorophenyl)-4,4-dimethylcyclopenten-1-yl)-4-(methylsulfonyl)benzene;

4-(2-(4-fluorophenyl)-4,4-dimethylcyclopenten-1-yl)benzenesulfonamide;

1-(2-(4-chlorophenyl)-4,4-dimethylcyclopenten-1-yl)-4-(methylsulfonyl)benzene;

4-(2-(4-chlorophenyl)-4,4-dimethylcyclopenten-1-yl)benzenesulfonamide;

4-(2-(4-fluorophenyl)cyclopenten-1-yl)benzenesulfonamide;

4-(2-(4-chlorophenyl)cyclopenten-1-yl)benzenesulfonamide;

1-(2-(4-methoxyphenyl)cyclopenten-1-yl)-4-(methylsulfonyl)benzene;

1-(2-(2,3-difluorophenyl)cyclopenten-1-yl)-4-(methylsulfonyl)benzene;

4-(2-(3-fluoro-4-methoxyphenyl)cyclopenten-1-yl)benzenesulfonamide;

1-(2-(3-chloro-4-methoxyphenyl)cyclopenten-1-yl)-4-(methylsulfonyl)benzene;

4-(2-(3-chloro-4-fluorophenyl)cyclopenten-1-yl)benzenesulfonamide;

4-(2-(2-methylpyridin-5-yl)cyclopenten-1-yl)benzenesulfonamide;

ethyl 2-(4-(4-fluorophenyl)-5-(4-(methylsulfonyl)phenyl)oxazol-2-yl)-2-benzyl-acetate;

2-(4-(4-fluorophenyl)-5-(4-(methylsulfonyl)phenyl)oxazol-2-yl)acetic acid;

2-(tert-butyl)-4-(4-fluorophenyl)-5-(4-(methylsulfonyl)phenyl)oxazole;

4-(4-fluorophenyl)-5-(4-(methylsulfonyl)phenyl)-2-phenyloxazole;

4-(4-fluorophenyl)-2-methyl-5-(4-(methylsulfonyl)phenyl)oxazole; and

4-(5-(3-fluoro-4-methoxyphenyl)-2-trifluoromethyl-4-oxazolyl)benzenesulfonamide;

or a pharmaceutically acceptable salt thereof.

NK-1 receptor antagonists of use in the present invention are described in published European Patent Specification Nos. 0 360 390, 0 394 989, 0 429 366, 0 443 132, 0 482 539, 0 512 901, 0 512 902, 0 514 273, 0 514 275, 0 517 589, 0 520 555, 0 522 808, 0 528 495, 0 532 456, 0 533 280, 0 536 817, 0 545 478, 0 577 394, 0 590 152, 0 599 538, 0 610 793, 0 634 402, 0 686 629, 0 693 489, 0 694 535, 0 699 655, 0 699 674, 0 707 006, 0 708 101, 0 714 891, 0 723 959, 0 733 632 and 0 776 893; and in International Patent Specification Nos. 90/05525, 90/05729, 91/09844, 91/18899, 92/01688, 92/06079, 92/12151, 92/15585, 92/17449, 92/20661, 92/20676, 92/21677, 93/00330, 93/00331, 93/01159, 93/01165, 93/01169, 93/01170, 93/06099, 93/09116, 93/10073, 93/14113, 93/18023, 93/19064, 93/21155, 9321181, 93/23380, 93/24465, 94/01402, 94/02461, 94/03429, 94/03445, 94/04494, 94/04496, 94/05625, 94/07843, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 96/22525, 95/23798, 95/26338, 95/28418, 95/30674, 95/30687, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942, 97/21702, 97/30055, 97/49710 and 98/01450; and in British Patent Specification Nos. 2 266 529, 2 268 931, 2, 269 170, 2 269 590, 2 271 774, 2 292 144, 2 293 168, 2293 169, 2 302 689 and 2 309 458.

Particularly preferred NK-1 receptor antagonists are those described in European Patent Specification No. 0 577 394, especially compounds of formula (I):

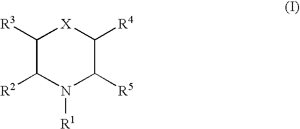

or a pharmaceutically acceptable salt thereof, wherein:

R¹ is selected from the group consisting of:

(1) C₁₋₆alkyl, substituted with one or more of the substituents selected from:

(a) heterocycle, wherein the heterocycle is selected from the group consisting of:

(A) benzimidazolyl,

(B) imidazolyl,

(C) isoxazolyl,

(D) isothiazolyl,

(E) oxadiazolyl,

(F) pyrazinyl,

(G) pyrazolyl,

(H) pyridyl,

(I) pyrrolyl,

(J) tetrazolyl,

(K) thiadiazolyl,

(L) triazolyl, and

(M) piperidinyl,

and wherein the heterocycle is unsubstituted or substituted with one or more substituent(s) selected from:

(i) C₁₋₆alkyl, unsubstituted or substituted with halo, —CF₃, —OCH₃, or phenyl,

(ii) C₁₋₆alkoxy,

(iii) oxo,

(iv) thioxo,

(v) cyano,

(vi) —SCH₃,

(vii) phenyl,

(viii) hydroxy,

(ix) trifluoromethyl,

(x) —(CH₂)ₘ—NR⁹R¹⁰, wherein m is 0, 1 or 2, and R⁹ and R¹⁰ are independently selected from:

(I) hydrogen,

(II) C₁₋₆alkyl,

(III) hydroxyC₁₋₆alkyl, and

(IV) phenyl,

(xi) —NR⁹COR¹⁰, wherein R⁹ and R¹⁰ are as defined above, and

(xii) —CONR⁹R¹⁰, wherein R⁹ and R¹⁰ are as defined above,

R² and R³ are independently selected from the group consisting of:

(1) hydrogen;

(2) C₁₋₆alkyl

(3) C₂₋₆alkenyl, and

(5) phenyl;

X is —O—;

R⁴ is

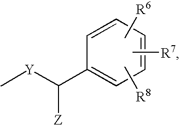

R⁵ is phenyl, unsubstituted or substituted with halo;

R⁶, R⁷ and R⁸ are independently selected from the group consisting of:

(1) hydrogen,

(2) C₁₋₆alkyl,

(3) halo, and

(4) —CF₃;

Y is —O—; and

Z is hydrogen or C₁₋₄alkyl;

and pharmaceutically acceptable salts thereof.

Particularly preferred compounds of formula (I) are:

4-(3-(1,2,4-triazolo)methyl)-2(S)-(3,5-bis(trifluoromethyl)benzyloxy)-3(S)-phenyl-morpholine;

4-(3-(1,2,4-triazolo)methyl)-2(S)-(3,5-bis(trifluoromethyl)benzyloxy)-3(R)-phenyl-morpholine;

4-(3-(5-oxo-1H,4H-1,2,4-triazolo)methyl)-2(S)-(3,5-bis(trifluoromethyl)benzyloxy)-3(S)-phenyl-morpholine; and

2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine; or a pharmaceutically acceptable salt thereof.

Further preferred NK-1 receptor antagonists are those described in International (PCT) Patent Specification No. WO 95/18124, especially compounds of formula (II) and pharmaceutically acceptable salts thereof:

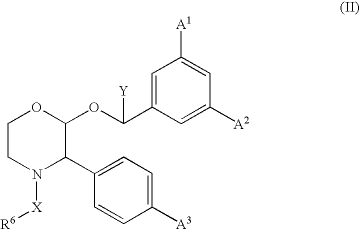

wherein:

A¹ is fluorine or CF₃;

A² is fluorine or CF₃;

A³ is fluorine or hydrogen;

R⁶ is a 5-membered or 6-membered heterocyclic ring containing 2 or 3 nitrogen atoms optionally substituted by ═O, ═S or a C₁₋₄alkyl group, and optionally substituted by a group of the formula ZNR⁷R⁸ where

Z is C₁₋₄alkylene or C₃₋₆cycloalkylene;

R⁷ is hydrogen, C₁₋₄alkyl, C₃₋₇cycloalkyl or C₃₋₇cycloalkylC₁₋₄alkyl, or C₂₋₄alkyl substituted by C₁₋₄alkoxy or hydroxyl;

R⁸ is hydrogen, C₁₋₄alkyl, C₃₋₇cycloalkyl or C₃₋₇cycloalkylC₁₋₄alkyl, or C₂₋₄alkyl substituted by one or two substituents selected from C₁₋₄alkoxy, hydroxyl or a 4, 5 or 6 membered heteroaliphatic ring containing one or two heteroatoms selected from N, O and S;

or R⁷, R⁸ and the nitrogen atom to which they are attached form a heteroaliphatic ring of 4 to 7 ring atoms, optionally substituted by a hydroxy group, and optionally containing a double bond, which ring may optionally contain an oxygen or sulphur ring atom, a group S(O) or S(O)₂ or a second nitrogen atom which will be part of a NH or NR^{c} moiety where R^{c} is C₁₋₄alkyl optionally substituted by hydroxy or C₁₋₄alkoxy;

or R⁷, R⁸ and the nitrogen atom to which they are attached form a non-aromatic azabicyclic ring system of 6 to 12 ring atoms;

or Z, R⁷ and the nitrogen atom to which they are attached form a heteroaliphatic ring of 4 to 7 ring atoms which may optionally contain an oxygen ring atom;

X is an alkylene chain of 1 to 4 carbon atoms optionally substituted by oxo; and

Y is a C₁₋₄alkyl group optionally substituted by a hydroxyl group;

with the proviso that if Y is C₁₋₄alkyl, R⁶ is susbstituted at least by a group of formula ZNR⁷R⁸ as defined above.

Particularly preferred compounds of formula (II) include:

2-(R)-(1-(R)-(3,5-bis(trifluoromethyl)phenyl)ethoxy)-4-(5-(dimethylamino) methyl-1,2,3-triazol-4-yl)methyl-3-(S)-phenylmorpholine;

2-(R)-(1-(R)-(3,5-bis(trifluoromethyl)phenyl)ethoxy)-4-(5-(dimethylamino) methyl-1,2,3-triazol-4-yl)methyl-3-(S)-(4-fluorophenyl)morpholine;

2-(R)-(1-(S)-(3,5-bis(trifluoromethyl)phenyl)-2-hydroxyethoxy)-3-(S)-(4-fluorophenyl)-4-(1,2,4-triazol-3-yl)methylmorpholine;

and pharmaceutically acceptable salts thereof.

Further preferred NK-1 receptor antagonists are those described in European Patent Specification No. WO 95/23798, especially compounds of formula (III):

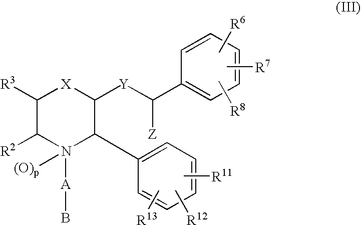

or a pharmaceutically acceptable salt thereof, wherein:

R² and R³ are independently selected from the group consisting of:

(1) hydrogen,

(2) C₁₋₆alkyl,

(3) C₂₋₆alkenyl, and

(4) phenyl;

R⁶, R⁷ and R⁸ are independently selected from the group consisting of:

(1) hydrogen,

(2) C₁₋₆alkyl,

(3) fluoro,

(4) chloro,

(5) bromo,

(6) iodo, and

(7) —CF₃;

R¹¹, R¹² and R¹³ are independently selected from the group consisting of:

(1) fluoro,

(2) chloro,

(3) bromo, and

(4) iodo;

A is unsubstituted ₁₋₆alkyl;

B is selected from the group consisting of:

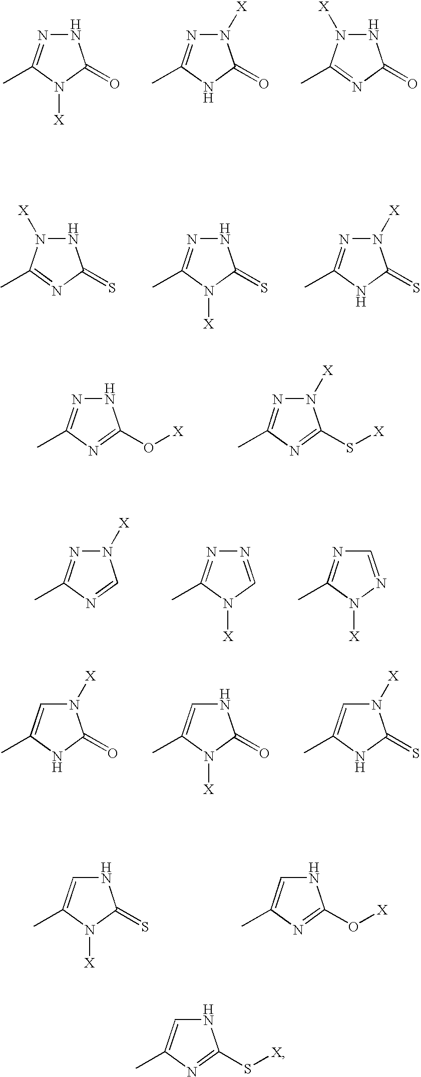

p is 0 or 1;

X is selected from:

(a) —PO(OH)O—.M⁺, wherein M⁺ is a pharmaceutically acceptable monovalent counterion,

(b) —PO(O—)₂.2M⁺,

(c) —PO(O—)₂.D²⁺, wherein D²⁺ is a pharmaceutically acceptable divalent counterion,

(d) —CH(R⁴)—PO(OH)O⁻.M⁺, wherein R⁴ is hydrogen or C₁₋₃alkyl,

(e) —CH(R⁴)—PO(O⁻)₂.2M⁺,

(f) —CH(R⁴)—PO(O⁻⁾₂.D²⁺,

(i) —CO—CH₂CH₂—CO₂⁻.M⁺,

(j) —CH(CH₃)—O—CO—R⁵, wherein R⁵ is selected from the group consisting of:

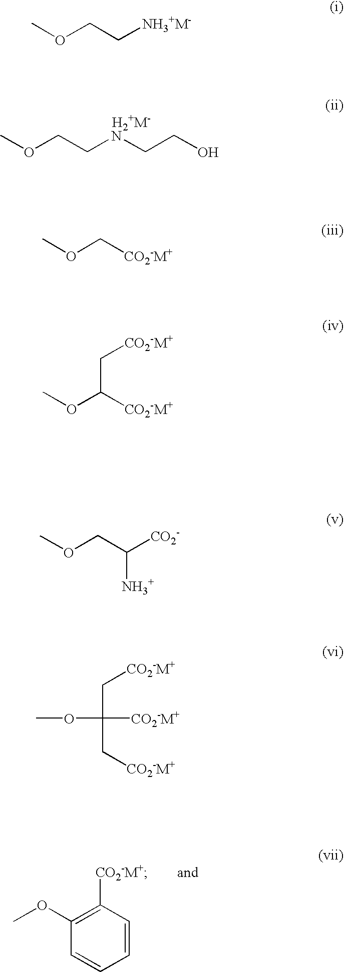

Y is −O—;

Z is hydrogen or C₁₋₆alkyl;

and pharmaceutically acceptable salts thereof

Particularly preferred compounds of formula (III) include:

(1) 2-(S)-(3,5-bis(trifluoromethyl)benzyloxy)-3-(S)-phenyl-4-(3-(5-oxo-1H,4H-1,2,4-triazolo)methyl)morpholine N-oxide;

(2) 2-(S)-(3,5-bis(trifluoromethyl)benzyloxy)-3-(S)-phenyl-4-(3-(4-(ethoxycarbonyloxy-1-ethyl)-5-oxo-1H-1,2,4-triazolo)methyl)morpholine;

(3) 2-(R)-(1-(R)-(3,5-bis(trifluoromethyl)phenyl)ethoxy)-3-(S)-(4-fluorophenyl)-4-(3-(4-monophosphoryl-5-oxo-1H-1,2,4-triazolo)methyl)morpholine;

(4) 2-(R)-(1-(R)-(3,5-bis(trifluoromethyl)phenyl)ethoxy)-3-(S)-(4-fluorophenyl)-4-(3-(1-monophosphoryl-5-oxo-1H-1,2,4-triazolo)methyl)morpholine;

(5) 2-(R)-(1-(R)-(3,5-bis(trifluoromethyl)phenyl)ethoxy)-3-(S)-(4-fluorophenyl)-4-(3-(2-monophosphoryl-5-oxo-1H-1,2,4-triazolo)methyl)morpholine;

(6) 2-(R)-(1-(R)-(3,5-bis(trifluoromethyl)phenyl)ethoxy)-3-(S)-(4-fluorophenyl)-4-(3-(5-oxyphosphoryl-1H-1,2,4-triazolo)methyl)morpholine;

(7) 2-(S)-(1-(R)-(3,5-bis(trifluoromethyl)phenyl)ethoxy)-3-(S)-(4-fluorophenyl)-4-(3-(1-monophosphoryl-5-oxo-4H-1,2,4-triazolo)methyl)morpholine;

and pharmaceutically acceptable salts thereof.

Further preferred NK-1 receptor antagonists are those described in International Patent Specification No. WO 97/49710, especially compounds of formula (IV):

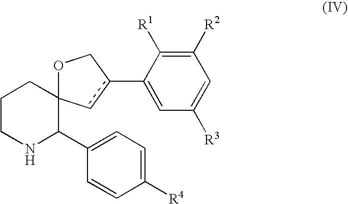

wherein

R¹ represents hydrogen, hydroxy, C₁₋₆alkyl, C₂₋₆alkenyl, C₃₋₇cycloalkyl, C₃₋₇cycloalkylC₁₋₄alkyl, C₁₋₆alkoxy, fluoroC₁₋₆alkoxy, C₁₋₆alkoxyC₁₋₄alkyl, C₁₋₆alkoxyC₁₋₄alkoxy, fluoroC₁₋₆alkoxyC₁₋₄alkyl, C₂₋₆alkenyloxy, C₃₋₇cycloalkoxy, C₃₋₇cycloalkylC₁₋₄alkoxy, phenoxy, benzyloxy, cyano, halogen, NR^{a}R^{b}, SR^{a}, SOR^{a}, SO₂R^{a}, OSO₂R^{a}, NR^{a}COR¹⁴, COR^{a}, CO₂R^{a} or CONR^{a}R^{b}where R^{a}and R^{b}each independently represent hydrogen, C₁₋₄alkyl or fluoroC₁₋₄alkyl;

R² represents hydrogen, halogen, C₁₋₆alkyl or C₁₋₆alkoxy;

or R¹ and R² may be joined together such that there is formed a 5- or 6-membered saturated or unsaturated ring containing one or two atoms selected from nitrogen, oxygen and sulphur, which ring is optionally substituted by a group selected from C₁₋₄alkyl, CF₃, ═O or ═S;

R³ represents hydrogen, halogen, C₁₋₆alkyl, fluoroC₁₋₆alkyl, C₁₋₆alkoxy, fluoroC₁₋₆alkoxy, C₃₋₇cycloalkyl, C₃₋₇cycloalkylC₁₋₄alkyl, cyano, SR^{a}, SOR^{a}, SO₂R^{a}, NR^{a}R^{b}, NR^{a}COR¹⁴, COR^{a}, CO₂R^{a}, CONR^{a}R^{b}or C₁₋₄alkyl substituted by cyano, CO₂R^{a}or CONR^{a}R^{b}where R^{a}and R^{b}are as previously defined;

R⁴ represents hydrogen, halogen, C₁₋₆alkyl, C₁₋₆alkoxy, CF₃, OCF₃, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy, where R^{a}and R^{b}are as previously defined; and

the broken line represents an optional double bond;

and pharmaceutically acceptable salts thereof.

Particularly preferred compounds of formula (IV) include:

(3R,5R,6S)-3-(2-methoxy-5-(trifluoromethoxy)phenyl)-6-phenyl-1-oxa-7-aza-spiro[4.5]decane;

(3R,5R,6S)-3-(2-methoxy-5-(trifluoromethyl)phenyl)-6-phenyl-1-oxa-7-aza-spiro[4.5]decane;

(3R,5R,6S)-7-benzyl-3-[2-methoxy-5-(trifluoromethoxy)phenyl]-6-phenyl-1-oxa-7-aza-spiro[4.5]decane;

(3R,5R,6S)-3-(2-methoxy-5-trifluoromethoxyphenyl)-6-phenyl-1-oxa-7-aza-spiro[4.5]decane;

(3R,5R,6S)-3,6-bis(phenyl)-1-oxa-7-aza-spiro[4.5]decane;

(3R,5R,6S)-7-benzyl-3-(2-methoxy-5-trifluoromethoxyphenyl)-6-phenyl 1-oxa-7-aza-spiro[4.5]decane;

(±)-(3R*,5R*,6S*)-3-(2-methoxyphenyl)-6-phenyl-1-oxa-7-(phenylmethoxycarbonyl)aza-spiro[4.5]decane;

(3R,5R,6S)-3-(2-methoxyphenyl)-6-phenyl-1-oxa-7-aza-spiro[4.5]decane;

(3S,5R,6S)-3-(2-cyclopropoxy-5-(trifluoromethoxy)phenyl)-6-phenyl-1-oxa-7-aza-spiro[4.5]decane;

(3R,5R,6S)-3-[2-cyclopropoxy-5-(trifluoromethoxy)phenyl]-6-phenyl-1-oxa-7-aza-spiro[4.5]decane;

(3S,5R,6S)-3-[2-cyclopropoxy-5-(trifluoromethyl)phenyl]-6-phenyl-1-oxa-7-aza-spiro[4.5]decane;

and pharmaceutically acceptable salts thereof.

Another class of NK-1 receptor antagonists of use in the present invention is that described in European Patent Specification No. 0 436 334, i.e. compounds of formula (V):

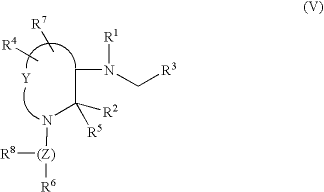

or a pharmaceutically acceptable salt thereof, wherein

Y is (CH₂)ₙ wherein n is an integer from 1 to 4, and wherein any one of the carbon-carbon single bonds in said (CH₂)ₙ may optionally be replaced by a carbon-carbon double bond, and wherein any one of the carbon atoms of said (CH₂)ₙ may optionally be substituted with R⁴, and wherein any one of the carbon atoms of said (CH₂)ₙ may optionally be substituted with R⁷;

Z is (CH₂)ₘ wherein in is an integer from 0 to 6, and wherein any one of the carbon-carbon single bonds of (CH₂)ₘ may optionally be replaced by a carbon-carbon double bond or a carbon-carbon triple bond, and any one of the carbon atoms of said (CH₂)ₘ may optionally be substituted with R⁸;

R¹ is hydrogen or C₁₋₈alkyl optionally substituted with hydroxy, C₁₋₄alkoxy or fluoro;

R² is a radical selected from hydrogen, C₁₋₆ straight or branched alkyl, C₃₋₇cycloalkyl wherein one of the CH₂ groups in said cycloalkyl may optionally be replaced by NH, oxygen or sulphur; aryl selected from phenyl and naphthyl; heteroaryl selected from indanyl, thienyl, furyl, pyridyl, thiazolyl, isothiazolyl, oxazolyl, isoxazolyl, triazolyl, tetrazolyl and quinolyl; phenyl-C₂₋₆alkyl, benzhydryl and benzyl, wherein each of said aryl and heteroaryl groups and the phenyl moieties of said benzyl, phenyl —C₂₋₆alkyl and benzhydryl may optionally be substituted with one or more substituents independently selected from halo, nitro, C₁₋₆ alkyl, C₁₋₆alkoxy, trifluoromethyl, amino, C₁₋₆alkylamino, C₁₋₆alkyl-O—CO, C₁₋₆alkyl-O—CO—C₁₋₆alkyl, C₁₋₆alkyl-CO—O, C₁₋₆alkyl-CO—C₁₋₆alkyl-O—, C₁₋₆alkyl-CO, C₁₋₆alkyl-CO—C₁₋₆alkyl-, di-C₁₋₆alkylamino, —CONH—C₁₋₆alkyl, C₁₋₆alkyl-CO—NH—C₁₋₆alkyl, —NHCOH and —NHCO—C₁₋₆alkyl; and wherein one of the phenyl moieties of said benzhydryl may optionally be replaced by naphthyl, thienyl, furyl or pyridyl;

R⁵ is hydrogen, phenyl or C₁₋₆alkyl;

or R² and R⁵ together with the carbon to which they are attached, form a saturated ring having from 3 to 7 carbon atoms wherein one of the CH₂ groups in said ring may optionally be replaced by oxygen, NH or sulfur;

R³ is aryl selected from phenyl and naphthyl; heteroaryl selected from indanyl, thienyl, furyl, pyridyl, thiazolyl, isothiazolyl, oxazolyl, isoxazolyl, triazolyl, tetrazolyl and quinolyl; and cycloalkyl having 3 to 7 carbon atoms wherein one of the (CH₂) groups in said cycloalkyl may optionally be replaced by NH, oxygen or sulphur;

wherein each of said aryl and heteroaryl groups may optionally be substituted with one or more substituents, and said C₃₋₇cycloalkyl may optionally be substituted with one or two substituents, each of said substituents being independently selected from halo, nitro, C₁₋₆alkyl, C₁₋₆alkoxy, trifluoromethyl, amino, C₁₋₆alkylamino, —CO—NH—C₁₋₆alkyl, C₁₋₆alkyl-CO—NH—C₁₋₆alkyl, —NHCOH and —NHCO—C₁₋₆alkyl;

R⁴ and R⁷ are each independently selected from hydroxy, halogen, halo, amino, oxo, cyano, methylene, hydroxymethyl, halomethyl, C₁₋₆alkylamino, di-C₁₋₁₆alkylamino, C₁₋₆alkoxy, C₁₋₆alkyl-O—CO, C₁₋₆alkyl-O—CO—C₁₋₆alkyl, C₁₋₆alkyl-CO—O, C₁₋₆alkyl-CO—C₁₋₆alkyl-O—, C₁₋₆alkyl-CO—, C₁₋₆alkyl-CO—C₁₋₆alkyl, and the radicals set forth in the definition of R²;

R⁶ is —NHCOR⁹, —NHCH₂R⁹, SO₂R⁸ or one of the radicals set forth in any of the definitions of R², R⁴ and R⁷;

R⁸ is oximino (═NOH) or one of the radicals set forth in any of the definitions of R², R⁴ and R⁷;

R⁹ is C₁₋₆alkyl, hydrogen, phenyl or phenylC₁₋₆alkyl;

with the proviso that (a) when m is 0, R⁸ is absent, (b) when R⁴, R⁶, R⁷ or R⁸ is as defined in R², it cannot form together with the carbon to which it is attached a ring with R⁵, and (c) when R⁴ and R⁷ are attached to the same carbon atom, then either each of R⁴ and R⁷ is independently selected from hydrogen, fluoro and C₁₋₆alkyl, or R⁴ and R⁷, together with the carbon to which they are attached, for a C₃₋₆ saturated carbocyclic ring that forms a spiro compound with the nitrogen-containing ring to which they are attached.

A particularly preferred compound of formula (V) is (2S,3S)-cis-3-(2-methoxybenzylamino)-2-phenylpiperidine; or a pharmaceutically acceptable salt thereof.

Another class of NK-1 receptor antagonists of use in the present invention is that described in International Patent Specification No. WO 93/21155, i.e. compounds of formula (VI):

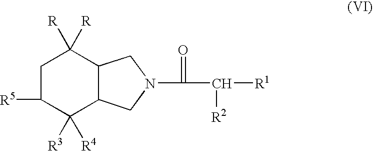

or a pharmaceutically acceptable salt thereof, wherein

radicals R are phenyl radicals optionally 2- or 3-substituted by a halogen atom or a methyl radical;

R¹ is optionally substituted phenyl, cyclohexadienyl, naphthyl, indenyl or optionally substituted heterocycle;

R² is H, halogen, OH, alkyl, aminoalkyl, alkylaminoalkyl, dialkylaminoalkyl, alkyloxy, alkylthio, acyloxy, carboxy, optionally substituted alkyloxycarbonyl, benzyloxycarbonyl, amino or acylamino;

R³ is optionally 2-substituted phenyl;

R⁴ is OH or fluorine when R⁵ is H;

or R⁴ and R⁵ are OH;

or R⁴ and R⁵ together form a bond.

A particularly preferred compound of formula (VI) is (3aS,4S,7aS)-7,7-diphenyl-4-(2-methoxyphenyl)-2-[(2S)-(2-methoxyphenyl)propionyl]perhydroisoindol-4-ol; or a pharmaceutically acceptable salt thereof.

Another class of NK-1 receptor antagonists of use in the present invention is that described in European Patent Specification No. 0 591 040, i.e. compounds of formula (VII):

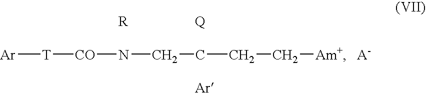

wherein

Ar represents an optionally substituted mono-, di- or tricyclic aromatic or heteroaromatic group;

T represents a bond, a hydroxymethylene group, a C₁₋₄alkoxymethylene group or a C₁₋₅alkylene group;

Ar′ represents a phenyl group which is unsubstituted or substituted by one or more substituents selected from halogen, preferably chlorine or fluorine, trifluoromethyl, C₁₋₄alkoxy, C₁₋₄alkyl where the said substituents may be the same or different; a thienyl group; a benzothienyl group; a naphthyl group; or an indolyl group;

R represents hydrogen, C₁₋₄alkyl, ω-C₁₋₄alkoxyC₁₋₄alkyl, or ω-C₂₋₄alkanoyloxyC₂₋₄alkyl;

Q represents hydrogen;

or Q and R together form a 1,2-ethylene, 1,3-propylene or 1,4-butylene group;

Am⁺ represents the radical

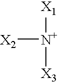

in which X₁, X₂ and X₃, together with the nitrogen atom to which they are attached, form an azabicyclic or azatricyclic ring system optionally substituted by a phenyl or benzyl group; and

A− represents a pharmaceutically acceptable anion.

A particularly preferred compound of formula (VII) is (+) 1-[2-[3-(3,4-dichlorophenyl)-1-[(3-isopropoxyphenyl)acetyl]-3-piperidinyl]ethyl]-4-phenyl-1-azabicyclo[2,2,2]octane; or a pharmaceutically acceptable salt, especially the chloride, thereof.

Another class of NK-1 receptor antagonists of use in the present invention is that described in European Patent Specification No. 0 532 456, i.e. compounds of formula (VIII):

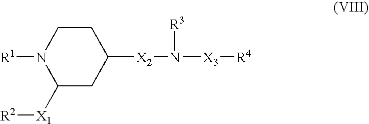

or a pharmaceutically acceptable salt thereof, wherein

R¹ represents an optionally substituted aralkyl, aryloxyalkyl, heteroaralkyl, aroyl, heteroaroyl, cycloalkylcarbonyl, aralkanoyl, heteroarylalkanoyl, aralkoxycarbonyl or arylcarbamoyl group or the acyl group of an α-amino acid optionally N-substituted by a lower alkanoyl or carbamoyl-lower alkanoyl group;

R² represents cycloalkyl or an optionally substituted aryl or heteroaryl group;

R³ represents hydrogen, alkyl, carbamoyl or an alkanoyl or alkenoyl group optionally substituted by carboxy or esterified or amidated carboxy;

R⁴ represents an optionally substituted aryl group or an optionally partially saturated heteroaryl group;

X₁ represents methylene, ethylene, a bond, an optionally ketalised carbonyl group or an optionally etherified hydroxymethylene group;

X₂ represents alkylene, carbonyl or a bond; and

X₃ represents carbonyl, oxo-lower alkyl, oxo(aza)-lower alkyl, or an alkyl group optionally substituted by phenyl, hydroxymethyl, optionally esterified or amidated carboxy, or (in other than the α-position) hydroxy.

A particularly preferred compound of formula (VIII) is (2R*,4S*)-2-benzyl-1-(3,5-dimethylbenzoyl)-N-(4-quinolinylmethyl)-4-piperidineamine;

or a pharmaceutically acceptable salt thereof.

Another class of NK-1 receptor antagonists of use in the present invention is that described in European Patent Specification No. 0 443 132, i.e. compounds of formula (IX)

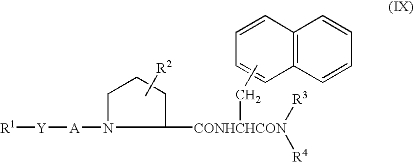

or a pharmaceutically acceptable salt thereof, wherein

R¹ is aryl, or a group of the formula:

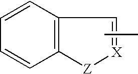

X is CH or N and

Z is O or N—R⁵, in which R⁵ is hydrogen or lower alkyl;

R² is hydroxy or lower alkoxy;

R³ is hydrogen or optionally substituted lower alkyl;

R⁴ is optionally substituted ar(lower)alkyl;

A is carbonyl or sulfonyl; and

Y is a bond or lower alkenylene.

A particularly preferred compound of formula (IX) is the compound of formula (IXa)

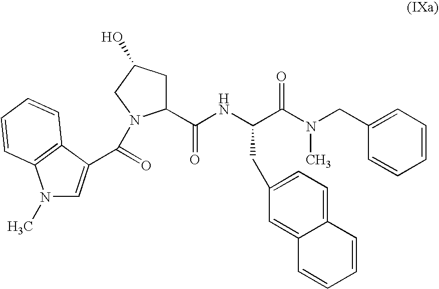

or a pharmaceutically acceptable salt thereof.

Another class of NK-1 receptor antagonists of use in the present invention is that described in International Patent Specification No. WO 92/17449, i.e. compounds of the formula (X)

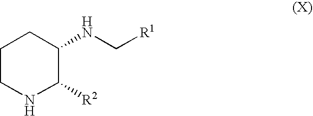

or a pharmaceutically acceptable salt thereof, wherein

R¹ is aryl selected from indanyl, phenyl and naphthyl; heteroaryl selected from thienyl, furyl, pyridyl and quinolyl; and cycloalkyl having 3 to 7 carbon atoms, wherein one of said carbon atoms may optionally be replaced by nitrogen, oxygen or sulfur; wherein each of said aryl and heteroaryl groups may optionally be substituted with one or more substituents, and said C₃₋₇cycloalkyl may optionally be substituted with one or two substituents, said substituents being independently selected from chloro, fluoro, bromo, iodo, nitro, C₁₋₁₀alkyl optionally substituted with from one to three fluoro groups, C₁₋₁₀alkoxy optionally substituted with from one to three fluoro groups, amino, C₁₋₁₀alkyl-S—, C₁₋₁₀alkyl-S(O)—, C₁₋₁₀alkyl-SO₂—, phenyl, phenoxy, C₁₋₁₀alkyl-SO₂NH—, C₁₋₁₀alkyl-SO₂NH—C₁₋₁₀alkyl-, C₁₋₁₀alkylamino-diC₁₋₁₀alkyl-, cyano, hydroxy, cycloalkoxy having 3 to 7 carbon atoms, C₁₋₆alkylamino, C₁₋₆dialkylamino, HC(O)NH— and C₁₋₁₀alkyl-C(O)NH—; and

R² is thienyl, benzhydryl, naphthyl or phenyl optionally substituted with from one to three substituents independently selected from chloro, bromo, fluoro, iodo, cycloalkoxy having 3 to 7 carbon atoms, C₁₋₁₀alkyl optionally substituted with from one to three fluoro groups and C₁₋₁₀alkoxy optionally substituted with from one to three fluoro groups.

A particularly preferred compound of formula (X) is (2S,3S)-3-(2-methoxy-5-trifluoromethoxybenzyl)-amino-2-phenylpiperidine; or a pharmaceutically acceptable salt thereof.

Another class of NK-1 receptor antagonists of use in the present invention is that described in International Patent Specification No. WO 95/08549, i.e. compounds of formula (XI)

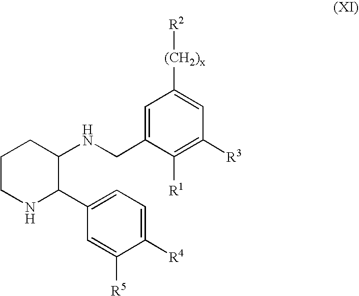

or a pharmaceutically acceptable salt thereof, wherein

R¹ is a C₁₋₄alkoxy group;

R² is

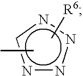

R³ is a hydrogen or halogen atom;

R⁴ and R⁵ may each independently represent a hydrogen or halogen atom, or a C₁₋₄alkyl, C₁₋₄alkoxy or trifluoromethyl group;

R⁶ is a hydrogen atom, a C₁₋₄alkyl, (CH₂)ₘcyclopropyl, —S(O)ₙC₁₋₄alkyl, phenyl, NR⁷R⁸, CH₂C(O)CF₃ or trifluoromethyl group;

R⁷ and R⁸ may each independently represent a hydrogen atom, or a C₁₋₄alkyl or acyl group;

x represents zero or 1;

n represents zero, 1 or 2; and

m represents zero or 1.

Particularly preferred compounds of formula (XI) are (2-methoxy-5-tetrazol-1-yl-benzyl)-([2S,3S]-2-phenyl-piperidin-3-yl)-amine; and [2-methoxy-5-(5-trifluoromethyl-tetrazol-1-yl)-benzyl]-([2S,3S]-2-phenyl-piperidin-3-yl)-amine; or a pharmaceutically acceptable salt thereof.

Another class of tachykinin antagonists of use in the present invention is that described in International Patent Specification No. WO 95/14017, i.e. compounds of formula (XII)

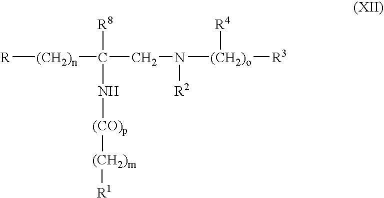

or a pharmaceutically acceptable salt thereof, wherein

m is zero, 1, 2 or 3;

n is zero or 1;

o is zero, 1 or 2;

p is zero or 1;

R is phenyl, 2- or 3-indolyl, 2- or 3-indolinyl, benzothienyl, benzofuranyl, or naphthyl;

which R groups may be substituted with one or two halo, C₁₋₃alkoxy, trifluoromethyl, C₁₋₄alkyl, phenyl-C₁₋₃alkoxy, or C₁₋₄alkanoyl groups;

R¹ is trityl, phenyl, diphenylmethyl, phenoxy, phenylthio, piperazinyl, piperidinyl, pyrrolidinyl, morpholinyl, indolinyl, indolyl, benzothienyl, hexamethyleneiminyl, benzofuranyl, tetrahydropyridinyl, quinolinyl, isoquinolinyl, reduced quinolinyl, reduced isoquinolinyl, phenyl-(C₁₋₄alkyl)-, phenyl-(C₁₋₄alkoxy)-, quinolinyl-(C₁₋₄alkyl)-, isoquinolinyl-(C₁₋₄alkyl)-, reduced quniolinyl-(C₁₋₄alkyl)-, reduced isoquinolinyl-(C₁₋₄alkyl)-, benzoyl-(C₁₋₃alkyl)-, C₁₋₄alkyl, or —NH—CH₂—R⁵;

any one of which R¹ groups may be substituted with halo, C₁₋₄alkyl, C₁₋₄alkoxy, trifluoromethyl, amino, C₁₋₄alkylamino, di(C₁₋₄alkyl)amino, or C₂₋₄alkanoylamino;

or any one of which R¹ groups may be substituted with phenyl, piperazinyl, C₃₋₈cycloalkyl, benzyl, C₁₋₄alkyl, piperidinyl, pyridinyl, pyrimidinyl, C₂₋₆alkanoylamino, pyrrolidinyl, C₂₋₆alkanoyl, or C₁₋₄alkoxycarbonyl;

any one of which groups may be substituted with halo, C₁₋₄alkyl, C₁₋₄alkoxy, trifluoromethyl, amino, C₁₋₄alkylamino, di(C₁₋₄alkyl)amino, or C₂₋₄alkanoylamino;

or R¹ is amino, a leaving group, hydrogen, C₁₋₄alkylamino, or di(C₁₋₄alkyl)amino;

R⁵ is pyridyl, anilino-(C₁₋₃alkyl)-, or anilinocarbonyl;

R² is hydrogen, C₁₋₄alkyl, C₁₋₄alkylsulfonyl, carboxy-(C₁₋₃alkyl)-, C₁₋₃alkoxycarbonyl-(C₁₋₃alkyl)-, or —CO—R⁶;

R⁶ is hydrogen, C₁₋₄alkyl, C₁₋₃haloalkyl, phenyl, C₁₋₃alkoxy, C₁₋₃hydroxyalkyl, amino, C₁₋₄alkylamino, di(C₁₋₄alkyl)amino, or —(CH₂)_{q}—R⁷;

q is zero to 3;

R⁷ is carboxy, C₁₋₄alkoxycarbonyl, C₁₋₄alkylcarbonyloxy, amino, C₁₋₄alkylamino, di(C₁₋₄alkyl)amino, C₁₋₆alkoxycarbonylamino, or phenoxy, phenylthio, piperazinyl, piperidinyl, pyrrolidinyl, morpholinyl, indolinyl, indolyl, benzothienyl, benzofuranyl, quinolinyl, phenyl-(C₁₋₄alkyl)-, quinolinyl-(C₁₋₄alkyl)-, isoquinolinyl-(C₁₋₄alkyl)-, reduced quinolinyl-(C₁₋₄alkyl)-, reduced isoquinolinyl-(C₁₋₄alkyl)-, benzoyl-C₁₋₃alkyl;

any one of which aryl or heterocyclic R⁷ groups may be substituted with halo, trifluoromethyl, C₁₋₄alkoxy, C₁₋₄alkyl, amino, C₁₋₄alkylamino, di(C₁₋₄alkyl)amino, or C₂₋₄alkanoylamino;

or any one of which R⁷ groups may be substituted with phenyl, piperazinyl, C₃₋₈cycloalkyl, benzyl, piperidinyl, pyridinyl, pyrimidinyl, pyrrolidinyl, C₂₋₆alkanoyl, or C₁₋₄alkoxycarbonyl;

any of which groups may be substituted with halo, trifluoromethyl, amino, C₁₋₄alkoxy, C₁₋₄alkyl, C₁₋₄alkylamino, di(C₁₋₄alkyl)amino- or C₂₋₄alkanoylamino;

R⁸ is hydrogen or C₁₋₆alkyl;

R³ is phenyl, phenyl-(C₁₋₆alkyl)-, C₃₋₈cycloalkyl, C₅₋₈cycloalkenyl, C₁₋₈alkyl, naphthyl, C₂₋₈alkenyl, or hydrogen;

any one or which groups except hydrogen may be substituted with one or two halo, C₁₋₃alkoxy, C₁₋₃alkylthio, nitro, trifluoromethyl, or C₁₋₃alkyl groups; and

R⁴ is hydrogen or C₁₋₃alkyl;

with the proviso that if R¹ is hydrogen or halo, R³ is phenyl, phenyl-(C₁₋₆alkyl)-, C₃₋₈cycloalkyl, C₅₋₈cycloalkenyl, or naphthyl.

A particularly preferred compound of formula (XII) is [N-(2-methoxybenzyl)acetylamino]-3-(1H-indol-3-yl)-2-[N-(2-(4-piperidin-1-yl)piperidin-1-yl)acetylamino]propane; or a pharmaceutically acceptable salt thereof.

The preferred compounds of formulae (I), (II) and (III) will have the 2- and 3-substituents on the morpholine ring in the cis arrangement, the preferred stereochemistry being as shown in the following general formula:

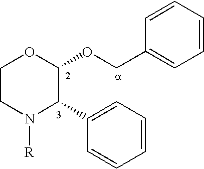

Where the benzyloxy moiety is α-substituted, the preferred stereochemistry of the α-carbon is either (R) when the substituent is an alkyl (e.g. methyl) group or (S) when the substituent is a hydroxyalkyl (e.g. hydroxymethyl) group.

The preferred compounds of formula (IV) will have the stereochemistry of the 5- and 6-positions as shown below (5-(R), 6-(S)). Where the optional double bond shown in formula (IV) is absent, the particularly preferred compounds will have the stereochemistry of the 3-position as shown below (3-(R)):

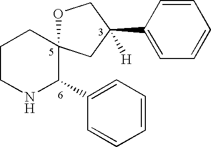

Unless otherwise defined herein, suitable alkyl groups include straight-chained and branched alkyl groups containing from 1 to 6 carbon atoms. Typical examples include methyl and ethyl groups, and straight-chained or branched propyl and butyl groups. Particular alkyl groups are methyl, ethyl, n-propyl, isopropyl, n-butyl, sec-butyl and tert-butyl.

Unless otherwise defined herein, suitable alkenyl groups include straight-chained and branched alkenyl groups containing from 2 to 6 carbon atoms. Typical examples include vinyl and allyl groups.

Unless otherwise defined herein, suitable alkynyl groups include straight-chained and branched alkynyl groups containing from 2 to 6 carbon atoms. Typical examples include ethynyl and propargyl groups.

Unless otherwise defined herein, suitable cycloalkyl groups include groups containing from 3 to 7 carbon atoms. Particular cycloalkyl groups are cyclopropyl and cyclohexyl.

Unless otherwise defined herein, suitable aryl groups include phenyl and naphthyl groups.

A particular aryl-C₁₋₆alkyl, e.g. phenyl-C₁₋₆alkyl, group is benzyl.

Unless otherwise defined herein, suitable heteroaryl groups include pyridyl, quinolyl, isoquinolyl, pyridazinyl, pyrimidinyl, pyrazinyl, pyranyl, furyl, benzofuryl, thienyl, benzthienyl, imidazolyl, oxadiazolyl and thiadiazolyl groups.

The term “halogen” as used herein includes fluorine, chlorine, bromine and iodine

Suitable pharmaceutically acceptable salts of the NK-1 receptor antagonists of use in the present invention include acid addition salts which may, for example, be formed by mixing a solution of the compound with a solution of a pharmaceutically acceptable non-toxic acid such as hydrochloric acid, fumaric acid, maleic acid, succinic acid, acetic acid, citric acid, tartaric acid, carbonic acid, phosphoric acid or sulphuric acid. Salts of amine groups may also comprise the quaternary ammonium salts in which the amino nitrogen atom carries an alkyl, alkenyl, alkynyl or aralkyl group. Where the compound carries an acidic group, for example a carboxylic acid group, the present invention also contemplates salts thereof, preferably non-toxic pharmaceutically acceptable salts thereof, such as the sodium, potassium and calcium salts thereof.

The compounds of use in this invention may have one or more chiral centers and the present compounds may occur as racemates, racemic mixtures and as individual diasteriomers or enantiomers with all such isomeric forms and mixtures thereof being included within the scope of this invention. Furthermore, some of the crystalline forms for compounds of the present invention may exist as polymorphs and as such are intended to be included in the present invention. In addition, some of the compounds of the instant invention may form solvates with water or common organic solvents. Such solvates and hydrates, as well as anhydrous compositions, are encompassed within the scope of this invention. Some of the compounds described herein may contain olefinic double bonds, and unless specified otherwise, are meant to include both E and Z geometric isomers.

The COX-2 inhibitors that may be used with this invention encompass all pharmaceutically acceptable salt forms of the compounds. Examples of such salt forms of COX-2 inhibitors include but are not limited to salts derived from inorganic bases including aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

The instant pharmaceutical combination comprising a COX-2 inhibitor in combination with a NK-1 receptor antagonist includes administration of a single pharmaceutical dosage formulation which contains both the COX-2 inhibitor and the NK-1 receptor antagonist, as well as administration of each active agent in its own separate pharmaceutical dosage formulation. Where separate dosage formulations are used, the COX-2 inhibitor and the NK-1 receptor antagonist can be administered at essentially the same time, i.e., concurrently, or at separately staggered times, i.e, sequentially. The instant pharmaceutical combination is understood to include all these regimens. Administration in these various ways are suitable for the present invention as long as the beneficial pharmaceutical effect of the COX-2 inhibitor and the NK-1 receptor antagonist are realized by the patient at substantially the same time. Such beneficial effect is preferably achieved when the target blood level concentrations of each active drug are maintained at substantially the same time. It is preferred that the COX-2 inhibitor and the NK-1 receptor antagonist be co-administered concurrently on a once-a-day dosing schedule; however, varying dosing schedules, such as the COX-2 inhibitor once, twice or more times per day and the NK-1 receptor antagonist once per day, is also encompassed herein. A single oral dosage formulation comprised of both the COX-2 inhibitor and the NK-1 receptor antagonist is preferred. A single dosage formulation will provide convenience for the patient, which is an important consideration especially for patients who already have an inflammatory disorder such as rheumatoid arthritis and may be in need of multiple medications.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. The dosage regimen utilizing a COX-2 inhibitor in combination with a NK-1 receptor antagonist is selected in accordance with a variety of factors including type, species, age, weight, sex and medical condition of the patient; the severity of the condition to be treated; the route of administration; the renal and hepatic function of the patient; and the particular compound or salt or ester thereof employed. Since two different active agents are being used together in a combination therapy, the potency of each of the agents and the interactive effects achieved by combining them together must also be taken into account. A consideration of these factors is well within the purview of the ordinarily skilled clinician for the purpose of determining the therapeutically effective or prophylactically effective dosage amounts needed to prevent, counter, or arrest the progress of the condition.

The term “patient” includes mammals, especially humans, who take a COX-2 inhibitor in combination with a NK-1 receptor antagonist for any of the uses described herein. Administering of the drug combination to the patient includes both self-administration and administration to the patient by another person.

The inhibitor of cyclooxygenase-2 may be administered at a dosage level up to conventional dosage levels for NSAIDs. Suitable dosage levels will depend upon the antiinflammatory effect of the chosen inhibitor of cyclooxygenase-2, but typically suitable levels will be about 0.001 to 50 mg/kg per day, preferably 0.005 to 30 mg/kg per day, and especially 0.05 to 10 mg/kg per day. The compound may be administered on a regimen of up to 6 times per day, preferably 1 to 4 times per day, and especially once per day.

A suitable dosage level for the NK-1 receptor antagonist is about 0.05 to 1500 mg per day, preferably about 0.25 to 1500 mg per day, and especially about 0.25 to 500 mg/kg per day. The compounds may be administered on a regimen of up to 6 times per day, preferably 1 to 4 times per day, especially 1 or 2 times daily.

The active agents employed in the instant combination therapy can be administered in such oral forms as tablets, capsules (each of which includes sustained release or timed release formulations), pills, powders, granules, elixirs, tinctures, suspensions, syrups, and emulsions. The instant invention includes the use of both oral rapid-release and time-controlled release pharmaceutical formulations. A particular example of an oral time-controlled release pharmaceutical formulation is described in U.S. Pat. No. 5,366,738. Oral formulations are preferred. Such pharmaceutical compositions are known to those of ordinary skill in the pharmaceutical arts; for example, see Remington's Pharmaceutical Sciences, Mack Publishing Co., Easton, Pa.

In the methods of the present invention, the active agents are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents and coloring and flavoring agents can also be incorporated into the mixture. Stabilizing agents such as antioxidants (BHA, BHT, propyl gallate, sodium ascorbate, citric acid) can also be added to stabilize the dosage forms. Other suitable components include gelatin, sweeteners, natural and synthetic gums such as acacia, tragacanth or alginates, carboxymethylcellulose, polyethylene glycol, waxes and the like.

The active drugs can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidyleholines.

Active drug may also be delivered by the use of monoclonal antibodies as individual carriers to which the compound molecules are coupled. Active drug may also be coupled with soluble polymers as targetable drug carriers. Such polymers can include polyvinyl-pyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoaciylates and cross linked or amphipathic block copolymers of hydrogels.

Although the active agents of the present method may be administered in divided doses, for example two or three times daily, a single daily dose of each of the COX-2 inhibitor and the NK-1 receptor antagonist is preferred, with a single daily dose of both agents in a single pharmaceutical composition being most preferred.

The instant invention also encompasses a process for preparing a pharmaceutical composition comprising combining the COX-2 inhibitor and the NK-1 receptor antagonist with a pharmaceutically acceptable carrier, as well as the pharmaceutical composition which is made by combining the COX-2 inhibitor and the NK-1 receptor antagonist with a pharmaceutically acceptable carrier.

A therapeutically effective amount of a COX-2 inhibitor and a NK-1 receptor antagonist can be used together for the preparation of a medicament useful for preventing or reducing the risk of developing an inflammatory disorder such as rheumatoid arthritis, halting or slowing the progression of an inflammatory disorder such as rheumatoid arthritis, once it has become clinically manifest, and preventing or reducing the risk of a first or subsequent occurrence of an inflammatory disorder such as rheumatoid arthritis. For example, the medicament may be comprised of a COX-2 inhibitor in combination with about 1 mg to 300 mg of a NK-1 receptor antagonist, or more particularly about 3 mg to 100 mg of the NK-1 receptor antagonist. More specific amounts of NK-1 receptor antagonist which may be used in the medicament preparation include 1 mg, 3 mg, 5 mg, 10 mg, 20 mg, 30 mg, 50 mg, 100 mg and 300 mg, as well as sub-milligram amounts of NK-1 receptor antagonists which have sufficient potency at such levels. As a further example, the medicament may be comprised of a NK-1 receptor antagonist, for example, at the above dosages, in combination with about 0.1 to 20 mg of a COX-2 inhibitor.

The instant invention also encompasses the use of a NK-1 receptor antagonist for the manufacture of a medicament for the combined use with a cyclooxygenase-2 inhibitor for preventing or reducing the risk of developing an inflammatory disorder, for halting or slowing the progression of an inflammatory disorder, or for preventing or reducing the risk of occurrence or recurrence of an inflammatory disorder; and the use of a cyclooxygenase-2 inhibitor for the preparation of a medicament for the combined use with a NK-1 receptor antagonist for preventing or reducing the risk of developing an inflammatory disorder, for halting or slowing the progression of an inflammatory disorder, or for preventing or reducing the risk of occurrence or recurrence of an inflammatory disorder.

The compounds of formulae (I), (II), (III), (IV), (V), (VI), (VII), (VIII), (IX), (X), (XI) and (XII) may be prepared by the methods described in EP-A-0 577 394 (or WO 95/16679), WO 95/18124, WO 95/23798, WO 97/49710, EP-A-0 436 334, WO 93/21155, EP-A-0 591 040, EP-A-0 532 456, EP-A-0 443 132, WO 92/17449, WO 95/08549 and WO 95/14017, respectively.

Particularly preferred NK-1 receptor antagonists of the formulae (I), (II), (III), (IV), (V), (VI), (VII), (VIII), (IX), (X), (XI) and (XII) for use in the present invention are compounds which are potent NK-1 receptor antagonists, i.e. compounds with an NK-1 receptor affinity (IC₅₀) of less than 100 nM. Most preferred are compounds of formulae (I), (II), (III) and (IV).

Even more preferred NK-1 receptor antagonists of use in the present invention are compounds which are potent NK-1 receptor antagonists with an NK-1 receptor affinity (IC₅₀) of less than 10 nM, favourably less than 2 nM and preferably less than 1 nM.

Especially preferred NK-1 receptor antagonists of use in the present invention are orally active, long acting, CNS-penetrant NK-1 receptor antagonists, identified using a combination of the following assays:

ASSAY 1: NK-1 Receptor Binding

NK-1 receptor binding assays are performed in intact Chinese hamster ovary (CHO) cells expressing the human NK-1 receptor using a modification of the assay conditions described by Cascieri et al, J. Pharmacol. Exp. Ther., 1992, 42, 458. Typically, the receptor is expressed at a level of 3×10⁵ receptors per cell. Cells are grown in monolayer culture, detached from the plate with enzyme-free dissociation solution (Speciality Media Inc.), and washed prior to use in the assay. ¹²⁵I-Tyr⁸-substance P(0.1 nM, 2000 Ci/mmol; New England Nuclear) is incubated in the presence or absence of test compounds (dissolved in 50 μl dimethylsulphoxide, DMSO) with 5×10⁴ CHO cells. Ligand binding is performed in 0.25 ml of 50 mM Tris-HCl, pH7.5, containing 5 mM MnCl₂, 150 mM NaCl, 0.02% bovine serum albumin (Sigma), 50 μg/ml chymostatin (Peninsula), 0.1 nM phenylmethylsulphonyl fluoride, 2 μg/ml pepstatin, 2 μg/ml leupeptin and 2.8 μg/ml furoyl saccharine. The incubation proceeds at room temperature until equilibrium is achieved (>40 minutes) and the receptor-ligand complex is harvested by filtration over GFJC filters pre-soaked in 0.1% polyethylenimine using a Tomtek 96-well harvester. Non-specific binding is determined using excess substance P (1 μM) and represents <10% of total binding.

ASSAY 2: Gerbil Foot-Tapping

CNS-penetrant NK-1 receptor antagonists for use in the present invention can be identified by their ability to inhibit foot tapping in gerbils induced by central infusion of NK-1 receptor agonists such as GR73632, based on the method of Rupniak & Williams, Eur. J. Pharmacol., 1994, 265, 179.

Male or female Mongolian gerbils (35-70 g) are anaesthetised by inhalation of an isoflurane/oxygen mixture to permit exposure of the jugular vein in order to permit administration of test compounds or vehicle in an injection volume of approximately 5 ml/kg i.v. Alternatively, test compounds may be administered orally or by subcutaneous or intraperitoneal routes. A skin incision is then made in the midline of the scalp to expose the skull. The selective NK-1 receptor agonist (e.g. GR73632 (d Ala[L-Pro⁹,Me-Leu¹⁰]-substance P-(7-11)) is infused directly into the cerebral ventricles (e.g. 3 pmol in 5 μl i.c.v., depending on test substance) by vertical insertion of a cuffed 27 gauge needle to a depth of 4.5 mm below bregma. The scalp incision is closed and the animal allowed to recover from anaesthesia in a clear perspex observation box (approximately 25 cm×20 cm×20 cm). The duration and/or intensity of hind foot tapping is then recorded continuously for approximately 5 minutes.

ASSAY 3: Ferret Emesis

Individually housed male ferrets (1.0-2.5 kg) are dosed orally by gavage with test compound. Ten minutes later they are fed with approximately 100 g of tinned cat food. At 60 minutes following oral dosing, cisplatin (10 mg/kg) is given i.v. via a jugular vein catheter inserted under a brief period of halothane anaesthesia. The catheter is then removed, the jugular vein ligated and the skin incision closed. The ferrets recover rapidly from the anaesthetic and are mobile within 10-20 minutes. The animals are observed continuously during recovery from the anaesthetic and for 4 hours following the cisplatin injection, after which time the animals are killed humanely. The numbers of retches and vomits occurring during the 4 hours after cisplatin administration are recorded by trained observers.

ASSAY 4: Footpad FCA Guinea Pig Model

Male Dunkin Hartly guinea pigs (180 g-200 g) are housed in groups in a twelve hour dark/light cycle and given vitamin C supplemented food and water ad libitum. Inflammation is induced by injection of 100 μl Freunds Complete Adjuvant (FCA) into the right hind footpad. The FCA is composed of 10 mg/ml heat killed mycobacterium blended with sterile paraffin. For the purposes of the study 6 animals are included in each treatment group and are dosed with experimental compounds as provided on a daily basis.

Inflammation is Assessed as Follows:

1. The diameter of the hind footpads is measured with vernier calipers at a consistent mid plantar site.

2. Measurement of thermal hyperalgesia is determined by the Hargreaves Method on unrestrained animals. The method determines the withdrawal latency to an infrared source (Ugo Basile). Hyperalgesia is assessed 4 hours after dosing.

3. Mechanical hyperalgesia is assessed using the Ugo Basile analgesy-meter according to the Randall-Selitto test. Force is applied onto the foot pad at a designated site increasing at 4.8 g per second. Hyperalgesia is assessed 4 hours after dosing.

A suitable selection cascade for NK₁ antagonists of use according to the present invention is as follows:

(i) Determine affinity for human NK₁ receptor in radioligand binding studies (Assay 1); select compounds with IC₅₀≦10 nM, preferably IC₅₀≦2 nM, especially IC₅₀≦1 nM.

(ii) Determine ability of compounds to penetrate CNS by their ability to inhibit foot tapping in gerbils induced by central injection of an NK₁ agonist (Assay 2); select compounds that inhibit foot tapping with ID₅₀≦3 mg/kg i.v., and preferably ID₅₀≦1 mg/kg iv. when administered immediately prior to central NK₁ agonist challenge, or ID₅₀≦30 mg/kg p.o., and preferably ID₅₀≦10 mg/kg p.o. 1 hour prior to challenge.

(iii) Determine central duration of action of compounds in gerbil foot tapping assay following intravenous administration 24 hours prior to central NK₁ agonist challenge; select compounds showing <25-fold loss of potency compared, with ID₅₀ determined in step (ii) above with the proviso that ID₅₀≦10 mg/kg i.v., and preferably ≦5 mg/kg i.v. after 24 hour pre-treatment.

(iv) Determine oral bioavailability of compounds by pharmacokinetic analysis, activity in gerbil foot tapping assay following oral administration and/or by ability to inhibit cisplatin-induced emesis in ferrets (Assay 3); select compounds with ID₅₀≦3 mg/kg p.o., and preferably ID₅₀≦1 mg/kg p.o.

Particularly preferred compounds of use in the present invention are identified using steps (i) to (iv) followed by step (v):

(v) Determine activity of compounds in assays for inhibition of pharmacologically evoked foot tapping in gerbils and/or inhibition of adjuvant arthritis in guinea-pigs (Assay 4). Select compounds with ID₅₀≦20 mg/kg, and preferably ID₅₀≦10 mg/kg.

Yet further preferred compounds of use in the present invention may be selected from those compounds which satisfy the NK-1 receptor binding criteria of step (i) which, in addition, have ≦5-fold shift in affinity when incubated in the presence of human serum albumin (HSA) to show non-specific protein binding.

One example of a NK-1 receptor antagonist of use in the present invention is the compound 2-(R)-(1-(R)-(3,5-bis(trifluoromethyl)phenyl)-ethoxy)-3-(S)-(4-fluorophenyl)-4-(3-(5-oxo-1H,4H-1,2,4-triazolo)methyl)-morpholine, the preparation of which is described in International Patent Specification No WO 95/16679. In the aforementioned assays, this compound has the following activity:

| | |
|---|---|
| human NK-1 receptor binding: | IC₅₀ = 0.1 nM |
| gerbil foot-tapping (5 mins.): | ID₅₀ = 0.36 mg/kg i.v. |
| gerbil foot-tapping (24 hrs.): | ID₅₀ = 0.33 mg/kg i.v. |
| ferret emesis: | ID₉₀ < 3 mg/kg p.o. |

Another example of a NK-1 receptor antagonist of use in the present invention is the compound 2-(R)-(1-(R)-(3,5-bis(trifluoromethyl)phenyl)ethoxy)-4-(5-(N,N-dimethylamino)methyl-1,2,3-triazol-4-yl)methyl-3-(S)-phenylmorpholine, the preparation of which is described in International Patent Specification No. WO 95/18124. In the aforementioned assays, this compound has the following activity:

| | |
|---|---|
| human NK-1 receptor binding: | IC₅₀ = 0.25 nM |
| gerbil foot-tapping (5 mins.): | ID₅₀ = 0.12 mg/kg i.v. |
| gerbil foot-tapping (24 hrs.): | ID₅₀ = 0.17 mg/kg i.v. |

Especially preferred COX-2 inhibitors of use in the present invention are identified using the following one or more of the following selection criteria:

(i) Determine the affinity for human COX-2 in whole cell assay; select compounds with ID₅₀≦40 nM, and preferably ID₅₀≦20 nM.

(ii) Determine oral bioavailability of compounds by pharmacokinetic analysis or inhibition of carrageenan-induced paw oedema in rats following oral administration. Select compounds with ED₅₀≦5 mg/kg p.o., and preferably ED₅₀≦2.5 mg/kg p.o.

(iii) Determine the ability of compounds to induce gastric ulceration or increase in faecal ⁵¹Cr excretion. Select compounds that have no adverse gastrointestinal effects at ≧100 mg/kg p.o., when administered up to two times a day.

(iv) Determine antinociceptive effects of compounds in carrageenan-induced hyperalgesia in rats; select compounds with ED₅₀≦5 mg/kg p.o., and preferably ED₅₀<2.5 mg/kg p.o.

(v) Determine the affinity for COX-2 in human whole blood assay which is used as an index for biochemical efficacy in the clinic and select compounds with —IC₅₀≦1 μM for inhibition of PGE₂.

The following examples illustrate pharmaceutical compositions according to the invention.

These formulations may be prepared with separate active ingredients or with a combination of active ingredients in one composition. In such combined preparations, the ratio of the COX-2 inhibitor and the NK-1 receptor antagonist will depend upon the choice of active ingredients.

### EXAMPLE 1

| | Amount (mg) per tablet | | |
|---|---|---|---|
| NK-1 receptor antagonist | 50.0 | 100.0 | 300.0 |
| Microcrystalline cellulose | 80.0 | 80.0 | 80.0 |
| Modified food corn starch | 80.0 | 80.0 | 80.0 |
| Lactose | 189.5 | 139.5 | 139.5 |
| Magnesium Stearate | 0.5 | 0.5 | 0.5 |

The active ingredient, cellulose, lactose and a portion of the corn starch are mixed and granulated with 10% corn starch paste. The resulting granulation is sieved, dried and blended with the remainder of the corn starch and the magnesium stearate. The resulting granulation is then compressed into tablets containing 50 mg, 100 mg and 300 mg of the NK-1 receptor antagonist per tablet.

### EXAMPLE 2

| | Amount (mg) per tablet | | |
|---|---|---|---|
| NK-1 receptor antagonist | 50.0 | 100.0 | 300.0 |
| COX-2 inhibitor | 20.0 | 20.0 | 20.0 |
| Microcrystalline cellulose | 80.0 | 80.0 | 80.0 |
| Modified food corn starch | 80.0 | 80.0 | 80.0 |
| Lactose | 169.5 | 119.5 | 119.5 |
| Magnesium Stearate | 0.5 | 0.5 | 0.5 |

The active ingredients, cellulose, lactose and a portion of the corn starch are mixed and granulated with 10% corn starch paste. The resulting granulation is sieved, dried and blended with the remainder of the corn starch and the magnesium stearate. The resulting granulation is then compressed into tablets containing 20 mg of the COX-2 inhibitor and 50 mg, 100 mg and 300 mg of the NK-1 receptor antagonist per tablet.

### EXAMPLE 3

Wet Granulated Tablet Composition

Amount (mg) per tablet

| | | | | |
|---|---|---|---|---|
| COX-2 Inhibitor | 25 | 12.5 | 10 | 5 |
| Microcrystalline cellulose | 79.7 | 86 | 87.2 | 89.7 |
| Lactose monohydrate | 79.7 | 86 | 87.2 | 89.7 |
| Hydroxypropyl cellulose | 6 | 6 | 6 | 6 |
| Croscarmellose sodium | 8 | 8 | 8 | 8 |
| Iron oxide | 0.6 | 0.6 | 0.6 | 0.6 |
| Magnesium stearate | 1 | 1 | 1 | 1 |

Tablet dose strengths of between 5 and 25 mg can be accomodated by varying total tablet weight, and the ratio of the first three ingredients. Generally it is preferable to maintain a 1:1 ratio for microcrystalline cellulose:lactose monohydrate.

### EXAMPLE 4

Directly Compressed Tablet Composition

Amount (mg) per tablet

| | | | | |
|---|---|---|---|---|
| COX-2 Inhibitor | 25 | 12.5 | 10 | 5 |
| Microcrystalline cellulose | 106.9 | 113.2 | 42.5 | 45 |
| Lactose anhydrate | 106.9 | 113.2 | 42.5 | 45 |
| Crosmellose sodium | 7.5 | 7.5 | 4 | 4 |
| Magnesium stearate | 3.7 | 3.7 | 1 | 1 |

Tablet dose strengths of between 5 and 25 mg can be accomodated by varying total tablet weight, and the ratio of the first three ingredients. Generally it is preferable to maintain a 1:1 ratio for microcrystalline cellulose:lactose monohydrate.

### EXAMPLE 5

Hard Gelatin Capsule Composition

| | Amount (mg) per capsule |
|---|---|
| COX-2 Inhibitor | 25 |
| Microcrystalline cellulose | 37 |
| Lactose anhydrate | 37 |
| Magnesium stearate | 1 |
| Hard gelatin capsule | 1 capsule |

Capsule dose strengths of between 1 and 50 mg can be accomodated by varying total fill weight, and the ratio of the first three ingredients. Generally it is preferable to maintain a 1:1 ratio for microcrystalline cellulose: lactose monohydrate.

### EXAMPLE 6

Oral Solution

| | Amount per 5 ml dose | |
|---|---|---|
| COX-2 Inhibitor | 50 | mg |
| Polyethylene oxide 400 | to 5 | ml |

Solution dose strengths of between 1 and 50 mg/5 ml can be accomodated by varying the ratio of the two ingredients.

### EXAMPLE 7

Oral Suspension

| | Amount per 5 ml dose | |
|---|---|---|
| COX-2 Inhibitor | 100 | mg |
| Polyvinylpyrrolidone | 150 | mg |
| Polyoxyethylene sorbitan monolaurate | 2.5 | mg |
| Benzoic acid | 10 | mg |
| sorbitol solution (70%) | to 5 | ml |

Suspension dose strengths of between 1 and 50 mg/5 ml can be accomodated by varying the ratio of the first two ingredients.

### EXAMPLE 8

Intravenous Infusion

| | Amount per 200 ml dose | |
|---|---|---|
| COX-2 inhibitor | 1 | mg |
| Polyethylene oxide 400 | 0.2 | mg |
| Sodium chloride | 1.8 | mg |
| Purified water | to 200 | ml |

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. Use of a COX-2 inhibitor and a NK-1 receptor antagonist for the manufacture of a medicament for the treatment or prevention of inflammatory disorders.

2. A pharmaceutical composition for the treatment or prevention of inflammatory disorders comprising a COX-2 inhibitor and a NK-1 receptor antagonist, together with at least one pharmaceutically acceptable carrier or excipient.

3. A product comprising a COX-2 inhibitor and a NK-1 receptor antagonist as a combined preparation for simultaneous, separate or sequential use in the treatment or prevention of inflammatory disorders.

4. A method for the treatment or prevention of inflammatory disorders, which method comprises administration to a patient in need of such treatment of an amount of a COX-2 inhibitor and an amount of a NK-1 receptor antagonist, such that together they give effective relief.

5. A use according to claim 1, a composition according to claim 2, a product according to claim 3 or a method according to claim 4 wherein the COX-2 inhibitor is selected from the classes of compounds described in U.S. Pat. Nos. 5,344,991, 5,380,738, 5,393,790, 5,409,944, 5,434,178, 5,436,265, 5,466,823, 5,474,995, 5,510,368, 5,536,752, 5,550,142, 5,552,422, 5,604,253, 5,604,260, and 5,639,780; and International Patent Publication Nos. WO 94/13635, WO 94/15932, WO 94/20480, WO 94/26731, WO 94/27980, WO 95/00501, WO 95/15316, WO 96/03387, WO 96/03388, WO 96/06840, WO 96/21667, WO 96/31509, WO 96/36623, WO 97/14691, and WO 97/16435.

6. A use according to claim 1, a composition according to claim 2, a product according to claim 3 or a method according to claim 4 wherein the COX-2 inhibitor is selected from:

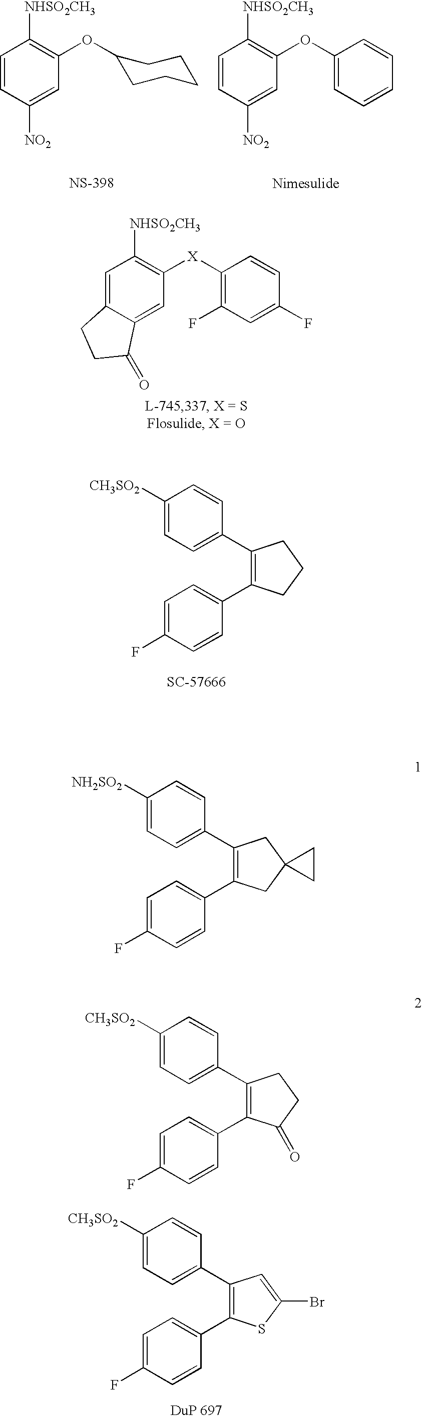

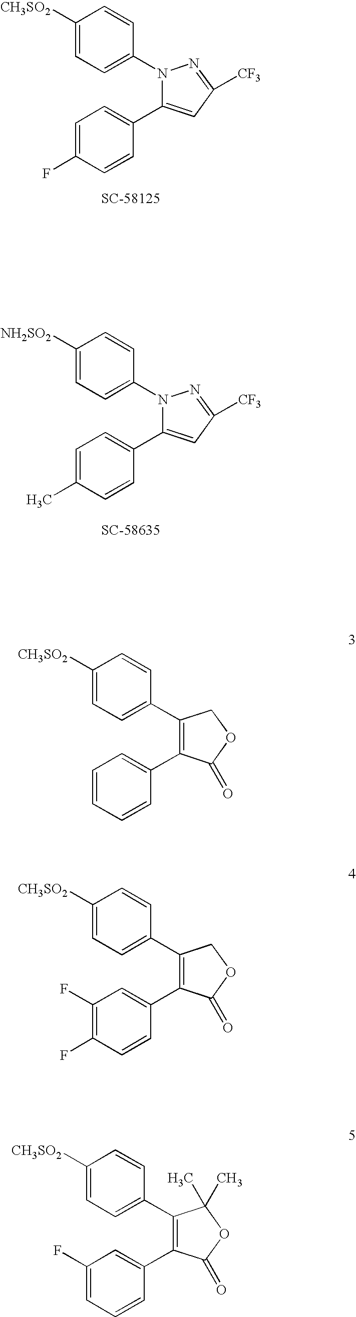

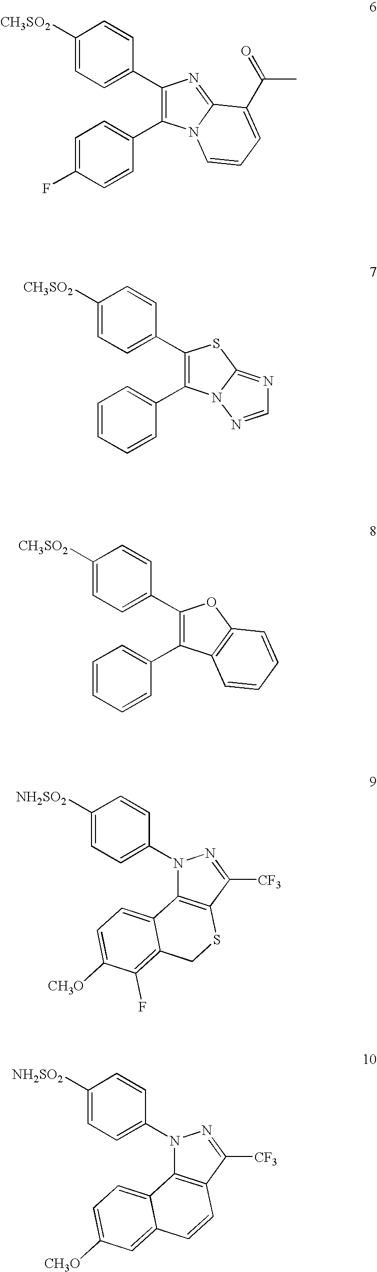

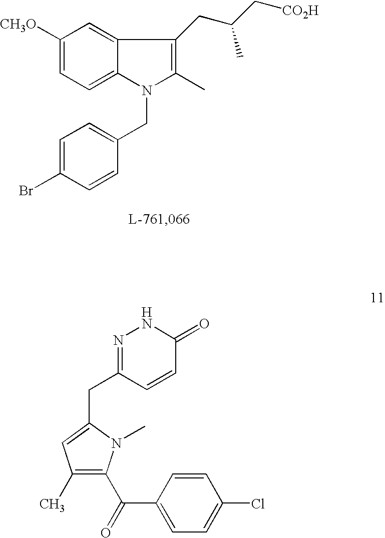

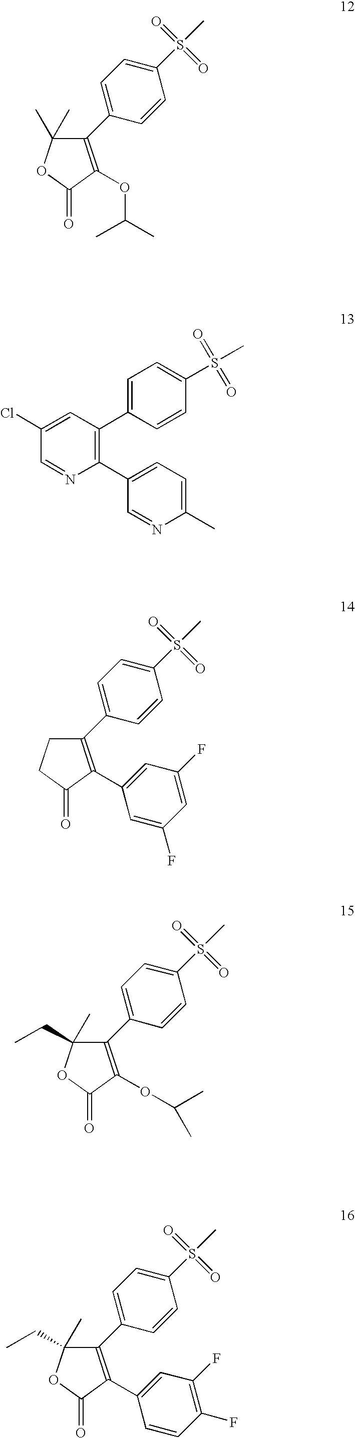

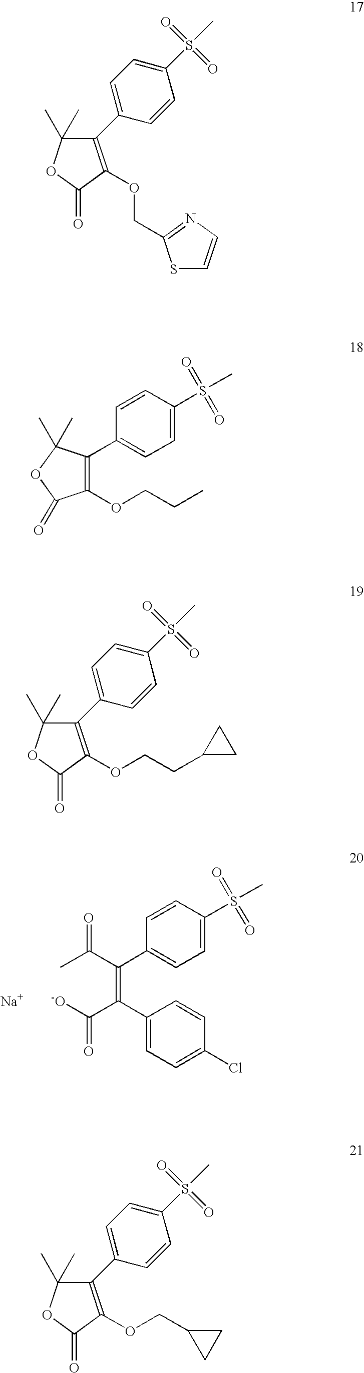

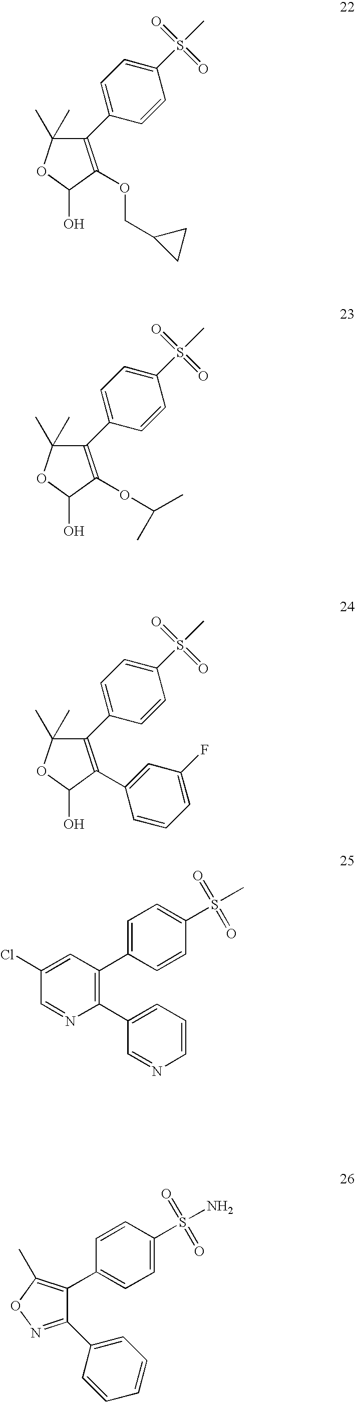

7. A use, composition, product or method according to claim 6 wherein the COX-2 inhibitor is selected from:
3: 3-phenyl-4-(4-(methylsulfonyl)phenyl)-2-(5H)-furanone;
4: 3-(3,4-difluorophenyl)-4-(4-(methylsulfonyl)phenyl)-2-(5H)-furanone;
5: 5,5-dimethyl-4-(4-(methylsulfonyl)phenyl)-3-(3-fluorophenyl)-5H-furan-2-one;
12: 5,5-dimethyl-4-(4-(methylsulfonyl)phenyl)-3-(2-propoxy)-5H-furan-2-one;
13: 5-chloro-3-(4-(methylsulfonyl)phenyl)-2-(2-methyl-5-pyridinyl)pyridine;
14: 2-(3,5-difluorophenyl)-3-(4-(methylsulfonyl)phenyl)-2-cyclopenten-1-one;
15: 5(S)-5-ethyl-5-methyl-4-(4-(methylsulfonyl)phenyl)-3-(2-propoxy)-5H-furan-2-one;
16: 5-ethyl-5-methyl-4-(4-(Methylsulfonyl)phenyl)-3-(3,4-difluorophenyl)-5H-furan-2-one;
17: 3-((2-thiazolyl)methoxy)-4-(4-(methylsulfonyl)phenyl)-5,5-dimethyl-5H-furan-2-one;
18: 3-propyloxy-4-(4-(methylsulfonyl)phenyl)-5,5-dimethyl-5H-furan-2-one;
19: 3-(1-cyclopropylethoxy)-5,5-dimethyl-4-(4-methylsulfonyl)phenyl)-5H-furan-2-one;
20: sodium 2-(4-chlorophenyl)-3-(4-(methylsulfonyl)phenyl)-4-oxo-2-pentenoate;
21: 3-(cyclopropylmethoxy)-5,5-dimethyl-4-(4-(methylsulfonyl)phenyl)-5H-furan-2-one;
22: 3-(cyclopropylmethoxy)-5,5-dimethyl-4-(4-(methylsulfonyl)phenyl)-2,5-dihydrofuran-2-ol;
23: 3-isopropoxy-5,5-dimethyl-4-(4-(methylsulfonyl)phenyl)-2,5-dihydrofuran-2-ol;
24: 5,5-dimethyl-3-(3-fluorophenyl)-2-hydroxy-4-(4-(methylsulfonyl)phenyl)-2,5-dihydrofuran;
25: 5-chloro-3-(4-(methylsulfonyl)phenyl)-2-(3-pyridinyl)pyridine;
26: 4-(5-methyl-3-phenyl-4-isoxazolyl)benzenesulfonamide;
or a pharmaceutically acceptable salt thereof.

8. A use according to claim 1, a composition according to claim 2, a product according to claim 3 or a method according to claim 4 wherein the COX-2 inhibitor is selected from:
5-(4-fluorophenyl)-[4-(methylsulfonyl)phenyl]-3-(trifluoromethyl)pyrazole;
4-(4-fluorophenyl)-5-[4-(methylsulfonyl)phenyl]-1-phenyl-3-(trifluoromethyl)pyrazole;
4-(5-(4-chlorophenyl)-3-(4-methoxyphenyl)-1H-pyrazol-1-yl)benzenesulfonamide;
4-(3,5-bis(4-methylphenyl)-1H-pyrazol-1-yl)benzenesulfonamide;
4-(5-(4-chlorophenyl)-3-phenyl-1H-pyrazol-1-yl)benzenesulfonamide;
4-(3,5-bis(4-methoxyphenyl)-1H-pyrazol-1-yl)benzenesulfonamide;
4-(5-(4-chlorophenyl)-3-(4-methylphenyl)-1H-pyrazol-1-yl)benzenesulfonamide;
4-(5-(4-chlorophenyl)-3-(4-nitrophenyl)-1H-pyrazol-1-yl)benzenesulfonamide;
4-(5-(4-chlorophenyl)-3-(5-chloro-2-thienyl)-1H-pyrazol-1-yl)benzenesulfonamide;
4-(4-chloro-3,5-diphenyl-1H-pyrazol-1-yl)benzenesulfonamide;
4-(5-(4-chlorophenyl)-3-(trifluoromethyl)-1H-pyrazol-1-yl)benzenesulfonamide;
4-(5-phenyl)-3-(trifluoromethyl)-1H-pyrazol-1-yl)benzenesulfonamide;
4-(5-(4-fluorophenyl)-3-(trifluoromethyl)-1H-pyrazol-1-yl)benzenesulfonamide;
4-(5-(4-methoxyphenyl)-3-(trifluoromethyl)-1H-pyrazol-1-yl)benzenesulfonamide;
4-(5-(4-chlorophenyl)-3-(difluoromethyl)-1H-pyrazol-1-yl)benzenesulfonamide;
4-(5-(4-methylphenyl)-3-(trifluoromethyl)-1H-pyrazol-1-yl)benzenesulfonamide;
4-(4-chloro-5-(4-chlorophenyl)-3-(trifluoromethyl)-1H-pyrazol-1-yl)benzenesulfonamide;
4-(3-(difluoromethyl)-5-(4-methylphenyl)-1H-pyrazol-1-yl)benzenesulfonamide;
4-(3-(difluoromethyl)-5-phenyl-1H-pyrazol-1-yl)benzenesulfonamide;
4-(3-(difluoromethyl)-5-(4-methoxyphenyl)-1H-pyrazol-1-yl)benzenesulfonamide;
4-(3-cyano-5-(4-fluorophenyl)-1H-pyrazol-1-yl)benzenesulfonamide;
4-(3-(difluoromethyl)-5-(3-fluoro-4-methoxyphenyl)-1H-pyrazol-1-yl)benzenesulfonamide;
4-(5-(3-fluoro-4-methoxyphenyl)-3-(trifluoromethyl)-1H-pyrazol-1-yl)benzenesulfonamide;
4-(4-chloro-5-phenyl-1H-pyrazol-1-yl)benzenesulfonamide;
4-(5-(4-chlorophenyl)-3-(hydroxyphenyl)-1H-pyrazol-1-yl)benzenesulfonamide;
4-(5-(4-(N,N-dimethylamino)phenyl)-3-(trifluoromethyl)-1H-pyrazol-1-yl)benzenesulfonamide;
5-(4-fluorophenyl)-6-(4-(methylsulfonyl)phenyl)spiro[2.4]hept-5-ene;
4-(6-(4-fluorophenyl)spiro[2.4]hept-5-en-5-yl)benzenesulfonamide;
6-(4-fluorophenyl)-7-(4-(methylsulfonyl)phenyl)spiro[3.4]oct-6-ene;
5-(3-chloro-4-methoxyphenyl)-6-(4-(methylsulfonyl)phenyl)spiro[2.4]hept-5-ene;
4-(6-(3-chloro-4-methoxyphenyl)spiro[2.4]hept-5-en-5-yl)benzenesulfonamide;
5-(3,5-dichloro-4-methoxyphenyl)-6-(4-(methylsulfonyl)phenyl)spiro[2.4]hept-5-ene;
5-(3-chloro-4-fluorophenyl)-6-(4-(methylsulfonyl)phenyl)spiro[2.4]hept-5-ene;
4-(6-(3,4-dichlorophenyl)spiro[2.4]hept-5-en-5-yl)benzenesulfonamide;
2-(3-chloro-4-fluorophenyl)-4-(4-fluorophenyl)-5-(4-methylsulfonylphenyl)thiazole;
2-(2-chlorophenyl)-4-(4-fluorophenyl)-5-(4-methylsulfonylphenyl)thiazole;
5-(4-fluorophenyl)-4-(4-methylsulfonylphenyl)-2-methylthiazole;
4-(4-fluorophenyl)-5-(4-methylsulfonylphenyl)-2-trifluoromethylthiazole;
4-(4-fluorophenyl)-5-(4-methylsulfonylphenyl)-2-(2-thienyl)thiazole;
4-(4-fluorophenyl)-5-(4-methylsulfonylphenyl)-2-benzylaminothiazole;
4-(4-fluorophenyl)-5-(4-methylsulfonylphenyl)-2-(1-propylamino)thiazole;
2-((3,5-dichlorophenoxy)methyl)-4-(4-fluorophenyl)-5-(4-(methylsulfonyl)phenyl)thiazole;
5-(4-fluorophenyl)-4-(4-methylsulfonylphenyl)-2-trifluoromethylthiazole;
1-methylsulfonyl-4-(1,1-dimethyl-4-(4-fluorophenyl)cyclopenta-2,4-dien-3-yl)benzene;
4-(4-(4-fluorophenyl)-1,1-dimethylcyclopenta-2,4-dien-3-yl)benzenesulfonamide;
5-(4-fluorophenyl)-6-(4-(methylsulfonyl)phenyl)spiro[2.4]hepta-4,6-diene 4-(6-(4-fluorophenyl)spiro[2.4]hepta-4,6-dien-5-yl)benzenesulfonamide;
6-(4-fluorophenyl)-2-methoxy-5-(4-(methylsulfonyl)phenyl)-pyridine-3-carbonitrile;
2-bromo-6-(4-fluorophenyl)-5-(4-(methylsulfonyl)phenyl)-pyridine-3-carbonitrile;
6-(4-fluorophenyl)-5-(4-(methylsulfonyl)phenyl)-2-phenyl-pyridine-3-carbonitrile;
4-(2-(4-methylpyridin-2-yl)-4-(trifluoromethyl)-1H-imidazol-1-yl)benzenesulfonamide;
4-(2-(5-methylpyridin-3-yl)-4-(trifluoromethyl)-1H-imidazol-1-yl)benzenesulfonamide;
4-(2-(2-methylpyridin-3-yl)-4-(trifluoromethyl)-1H-imidazol-1-yl)benzenesulfonamide;
3-(1-(4-(methylsulfonyl)phenyl)-4-(trifluoromethyl)-1H-imidazol-2-yl)benzenesulfonamide;
2-(1-(4-(methylsulfonyl)phenyl)-4-(trifluoromethyl)-1H-imidazol-2-yl)pyridine;
2-methyl-4-(1-(4-(methylsulfonyl)phenyl)-4-(trifluoromethyl)-1H-imidazol-2-yl)pyridine;
2-methyl-6-(1-(4-(methylsulfonyl)phenyl)-4-(trifluoromethyl)-1H-imidazol-2-yl)pyridine;
4-(2-(6-methylpyridin-3-yl)-4-(trifluoromethyl)-1H-imidazol-1-yl)benzenesulfonamide;
2-(3,4-difluorophenyl)-1-(4-(methylsulfonyl)phenyl)-4-(trifluoromethyl)-1H-imidazole;
4-(2-(4-methylphenyl)-4-(trifluoromethyl)-1H-imidazol-1-yl)benzenesulfonamide;
2-(4-chlorophenyl)-1-(4-(methylsulfonyl)phenyl)-4-methyl-1H-imidazole;
2-(4-chlorophenyl)-1-(4-(methylsulfonyl)phenyl)-4-phenyl-1H-imidazole;
2-(4-chlorophenyl)-4-(4-fluorophenyl)-1-(4-(methylsulfonyl)phenyl)-1H-imidazole;
2-(3-fluoro-4-methoxyphenyl)-1-(4-(methylsulfonyl)phenyl)-4-(trifluoromethyl)-1H-imidazole;
1-(4-(methylsulfonyl)phenyl)-2-phenyl-4-trifluoromethyl-1H-imidazole;
2-(4-methylphenyl)-1-(4-(methylsulfonyl)phenyl)-4-trifluoromethyl-1H-imidazole;
4-(2-(3-chloro-4-methylphenyl)-4-(trifluoromethyl)-1H-imidazol-1-yl)benzenesulfonamide;
2-(3-fluoro-5-methylphenyl)-1-(4-(methylsulfonyl)phenyl)-4-(trifluoromethyl)-1H-imidazole;
4-(2-(3-fluoro-5-methylphenyl)-4-(trifluoromethyl)-1H-imidazol-1-yl)benzenesulfonamide;
2-(3-methylphenyl)-1-(4-(methylsulfonyl)phenyl)-4-(trifluoromethyl)-1H-imidazole;
4-(2-(3-methylphenyl)-4-(trifluoromethyl)-1H-imidazol-1-yl)benzenesulfonamide;
1-(4-(methylsulfonyl)phenyl)-2-(3-chlorophenyl)-4-(trifluoromethyl)-1H-imidazole;
4-(2-(3-chlorophenyl)-4-(trifluoromethyl)-1H-imidazol-1-yl)benzenesulfonamide;
4-(2-phenyl-4-(trifluoromethyl)-1H-imidazol-1-yl)benzenesulfonamide;
4-(2-(4-methoxy-3-chlorophenyl)-4-(trifluoromethyl)-1H-imidazol-1-yl)benzenesulfonamide;
1-allyl-4-(4-fluorophenyl)-3-(4-(methylsulfonyl)phenyl)-5-(trifluoromethyl)-1H-pyrazole;
4-(1-ethyl-4-(4-fluorophenyl)-5-(trifluoromethyl)-1H-pyrazol-3-yl)benzenesulfonamide;
N-phenyl-(4-(4-fluorophenyl)-3-(4-(methylsulfonyl)phenyl)-5-(trifluoromethyl)-1H-pyrazol-1-yl)acetamide;
ethyl(4-(4-fluorophenyl)-3-(4-(methylsulfonyl)phenyl)-5-(trifluoromethyl)-1H-pyrazol-1-yl)acetate;
4-(4-fluorophenyl)-3-(4-(methylsulfonyl)phenyl)-1-(2-phenylethyl)-1H-pyrazole;
4-(4-fluorophenyl)-3-(4-(methylsulfonyl)phenyl)-1-(2-phenylethyl)-5-(trifluoromethyl)pyrazole;
1-ethyl-4-(4-fluorophenyl)-3-(4-(methylsulfonyl)phenyl)-5-(trifluoromethyl)-1H-pyrazole;
5-(4-fluorophenyl)-4-(4-(methylsulfonyl)phenyl)-2-(trifluoromethyl)-1H-imidazole;
4-(4-(methylsulfonyl)phenyl)-5-(2-thiophenyl)-2-(trifluoromethyl)-1H-imidazole;
5-(4-fluorophenyl)-2-methoxy-4-(4-(methylsulfonyl)phenyl)-6-(trifluoromethyl)pyridine;
2-ethoxy-5-(4-fluorophenyl)-4-(4-(methylsulfonyl)phenyl)-6-(trifluoromethyl)pyridine;
5-(4-fluorophenyl)-4-(4-(methylsulfonyl)phenyl)-2-(2-propynyloxy)-6-(trifluoromethyl)pyridine;
2-bromo-5-(4-fluorophenyl)-4-(4-(methylsulfonyl)phenyl)-6-(trifluoromethyl)pyridine;
4-(2-(3-chloro-4-methoxyphenyl)-4,5-difluorophenyl)benzenesulfonamide;
1-(4-fluorophenyl)-2-(4-(methylsulfonyl)phenyl)benzene;
5-difluoromethyl-4-(4-(methylsulfonyl)phenyl)-3-phenylisoxazole;
4-(3-ethyl-5-phenylisoxazol-4-yl)benzenesulfonamide;
4-(5-difluoromethyl-3-phenylisoxazol-4-yl)benzenesulfonamide;
4-(5-hydroxymethyl-3-phenylisoxazol-4-yl)benzenesulfonamide;
4-(5-methyl-3-phenylisoxazol-4-yl)benzenesulfonamide;
1-(2-(4-fluorophenyl)cyclopenten-1-yl)-4-(methylsulfonyl)benzene;
1-(2-(4-fluoro-2-methylphenyl)cyclopenten-1-yl)-4-(methylsulfonyl)benzene;
1-(2-(4-chlorophenyl)cyclopenten-1-yl)-4-(methylsulfonyl)benzene;
1-(2-(2,4-dichlorophenyl)cyclopenten-1-yl)-4-(methylsulfonyl)benzene;
1-(2-(4-trifluoromethylphenyl)cyclopenten-1-yl)-4-(methylsulfonyl)benzene;
1-(2-(4-methyl thiophenyl)cyclopenten-1-yl)-4-(methylsulfonyl)benzene;
1-(2-(4-fluorophenyl)-4,4-dimethylcyclopenten-1-yl)-4-(methylsulfonyl)benzene;
4-(2-(4-fluorophenyl)-4,4-dimethylcyclopenten-1-yl)benzenesulfonamide;
1-(2-(4-chlorophenyl)-4,4-dimethylcyclopenten-1-yl)-4-(methylsulfonyl)benzene;
4-(2-(4-chlorophenyl)-4,4-dimethylcyclopenten-1-yl)benzenesulfonamide;
4-(2-(4-fluorophenyl)cyclopenten-1-yl)benzenesulfonamide;
4-(2-(4-chlorophenyl)cyclopenten-1-yl)benzenesulfonamide;
1-(2-(4-methoxyphenyl)cyclopenten-1-yl)-4-(methylsulfonyl)benzene;
1-(2-(2,3-difluorophenyl)cyclopenten-1-yl)-4-(methylsulfonyl)benzene;
4-(2-(3-fluoro-4-methoxyphenyl)cyclopenten-1-yl)benzenesulfonamide;
1-(2-(3-chloro-4-methoxyphenyl)cyclopenten-1-yl)-4-(methylsulfonyl)benzene;
4-(2-(3-chloro-4-fluorophenyl)cyclopenten-1-yl)benzenesulfonamide;
4-(2-(2-methylpyridin-5-yl)cyclopenten-1-yl)benzenesulfonamide;
ethyl 2-(4-(4-fluorophenyl)-5-(4-(methylsulfonyl)phenyl)oxazol-2-yl)-2-benzyl-acetate;
2-(4-(4-fluorophenyl)-5-(4-(methylsulfonyl)phenyl)oxazol-2-yl)acetic acid;
2-(tert-butyl)-4-(4-fluorophenyl)-5-(4-(methylsulfonyl)phenyl)oxazole;
4-(4-fluorophenyl)-5-(4-(methylsulfonyl)phenyl)-2-phenyloxazole;
4-(4-fluorophenyl)-2-methyl-5-(4-(methylsulfonyl)phenyl)oxazole; and
4-(5-(3-fluoro-4-methoxyphenyl)-2-trifluoromethyl-4-oxazolyl)benzenesulfonamide;
or a pharmaceutically acceptable salt thereof.

9. A use according to claim 1, a composition according to claim 2, a product according to claim 3 or a method according to claim 4 wherein the NK-1 receptor antagonist is a compound of formula I: 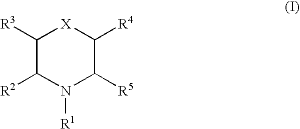
wherein:
R¹ is selected from the group consisting of:
(1) C₁₋₆alkyl, substituted with one or more of the substituents selected from:
(a) heterocycle, wherein the heterocycle is selected from the group consisting of:
(A) benzimidazolyl,
(B) imidazolyl,
(C) isoxazolyl,
(D) isothiazolyl,
(E) oxadiazolyl,
(F) pyrazinyl,
(G) pyrazolyl,
(H) pyridyl,
(I) pyrrolyl,
(J) tetrazolyl,
(K) thiadiazolyl,
(L) triazolyl, and
(M) piperidinyl,
and wherein the heterocycle is unsubstituted or substituted with one or more substituent(s) selected from:
(i) C₁₋₆alkyl, unsubstituted or substituted with halo, —CF₃, —OCH₃, or phenyl,
(ii) C₁₋₆alkoxy,
(iii) oxo,
(iv) thioxo,
(v) cyano,
(vi) —SCH₃,
(vii) phenyl,
(viii) hydroxy,
(ix) trifluoromethyl,
(x) —(CH₂)ₘ—NR⁹R¹⁰, wherein m is 0, 1 or 2, and R⁹ and R¹⁰ are independently selected from:
(I) hydrogen,
(II) C₁₋₆alkyl,
(III) hydroxyC₁₋₆alkyl, and
(IV) phenyl,
(xi) —NR⁹COR¹⁰, wherein R⁹ and R¹⁰ are as defined above, and
(xii) —CONR⁹R¹⁰, wherein R⁹ and R¹⁰ are as defined above,
R² and R³ are independently selected from the group consisting of:
(1) hydrogen;
(2) C₁₋₆alkyl
(3) C₂₋₆alkenyl, and
(5) phenyl;
X is —O—;
R⁴ is 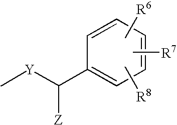
R⁵ is phenyl, unsubstituted or substituted with halo;
R⁶, R⁷ and R⁸ are independently selected from the group consisting of
(1) hydrogen,
(2) C₁₋₆alkyl,
(3) halo, and
(4) —CF₃;
Y is —O—; and
Z is hydrogen or C₁₋₄alkyl;
or a pharmaceutically acceptable salt thereof.

10. A use according to claim 1, a composition according to claim 2, a product according to claim 3 or a method according to claim 4 wherein the NK-1 receptor antagonist is a compound of formula II: 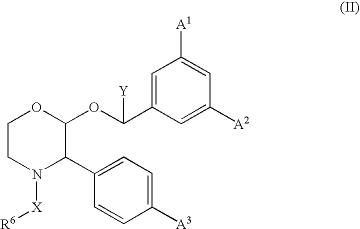
wherein:
A¹ is fluorine or CF₃;
A² is fluorine or CF₃;
A³ is fluorine or hydrogen;
R⁶ is a 5-membered or 6-membered heterocyclic ring containing 2 or 3 nitrogen atoms optionally substituted by ═O, ═S or a C₁₋₄alkyl group, and optionally substituted by a group of the formula ZNR⁷R⁸ where
Z is C₁₋₆alkylene or C₃₋₆cycloalkylene;
R⁷ is hydrogen, C₁₋₄alkyl, C₃₋₇cycloalkyl or C₃₋₇cycloalkylC₁₋₄alkyl, or C₂₋₄alkyl substituted by C₁₋₄alkoxy or hydroxyl;
R⁸ is hydrogen, C₁₋₄alkyl, C₃₋₇cycloalkyl or C₃₋₇cycloalkylC₁₋₄alkyl, or C₂₋₄alkyl substituted by one or two substituents selected from C₁₋₄alkoxy, hydroxyl or a 4, 5 or 6 membered heteroaliphatic ring containing one or two heteroatoms selected from N, O and S;
or R⁷, R⁸ and the nitrogen atom to which they are attached form a heteroaliphatic ring of 4 to 7 ring atoms, optionally substituted by a hydroxy group, and optionally containing a double bond, which ring may optionally contain an oxygen or sulphur ring atom, a group S(O) or S(O)₂ or a second nitrogen atom which will be part of a NH or NR^{c} moiety where R^{c} is C₁₋₄alkyl optionally substituted by hydroxy or C₁₋₄alkoxy;
or R⁷, R⁸ and the nitrogen atom to which they are attached form a non-aromatic azabicyclic ring system of 6 to 12 ring atoms;
or Z, R⁷ and the nitrogen atom to which they are attached form a heteroaliphatic ring of 4 to 7 ring atoms which may optionally contain an oxygen ring atom;
X is an alkylene chain of 1 to 4 carbon atoms optionally substituted by oxo; and
Y is a C₁₋₄alkyl group optionally substituted by a hydroxyl group;
with the proviso that if Y is C₁₋₄alkyl, R⁶ is susbstituted at least by a group of formula ZNR⁷R⁸ as defined above;
or a pharmaceutically acceptable salt thereof.

11. A use according to claim 1, a composition according to claim 2, a product according to claim 3 or a method according to claim 4 wherein the NK-1 receptor antagonist is a compound of formula III: 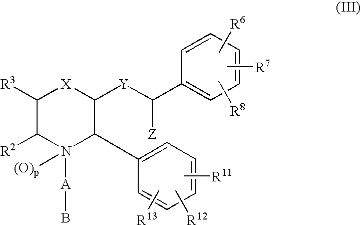
wherein:
R² and R³ are independently selected from the group consisting of:
(1) hydrogen,
(2) C₁₋₆alkyl,
(3) C₂₋₆alkenyl, and
(4) phenyl;
R⁶, R⁷ and R⁸ are independently selected from the group consisting of:
(1) hydrogen,
(2) C₁₋₆alkyl,
(3) fluoro,
(4) chloro,
(5) bromo,
(6) iodo, and
(7) —CF₃;
R¹¹, R¹² and R¹³ are independently selected from the group consisting of:
(1) fluoro,
(2) chloro,
(3) bromo, and
(4) iodo;
A is unsubstituted ₁₋₆alkyl;
B is selected from the group consisting of: 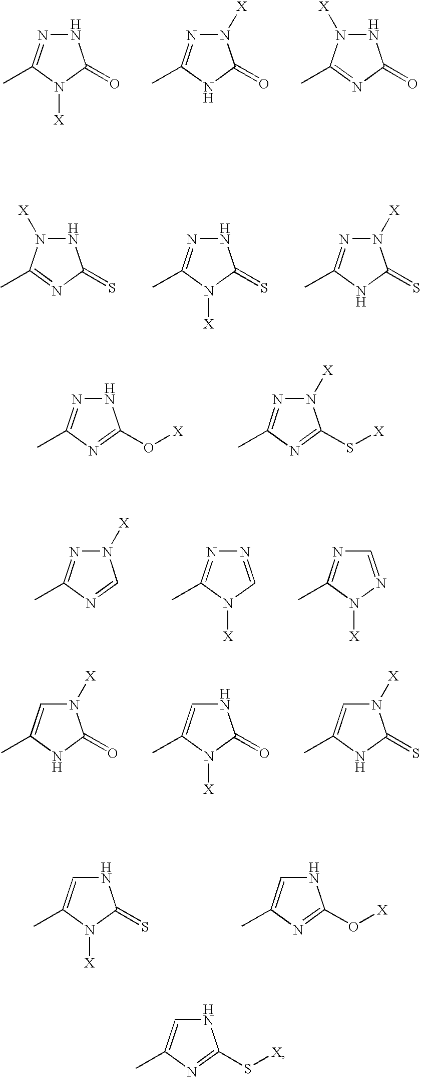
p is 0 or 1;
X is selected from:
(a) —PO(OH)O—.M⁺, wherein M⁺ is a pharmaceutically acceptable monovalent counterion.
(b) —PO(O)₂.2M⁺,
(c) —PO(O)₂.D²⁺, wherein D²⁺ is a pharmaceutically acceptable divalent counterion,
(d) —CH(R⁴)—PO(OH)O⁻*M⁺, wherein R⁴ is hydrogen or C₁₋₃alkyl,
(e) —CH(R⁴)—PO(O⁻)₂.2M⁺,
(f) —CH(R⁴)—PO(O⁻)₂.D²⁺,
(i) —CO—CH₂CH₂—CO₂⁻.M⁺,
(j) —CH(CH₃)—O—CO—R⁵, wherein R⁵ is selected from the group consisting of: 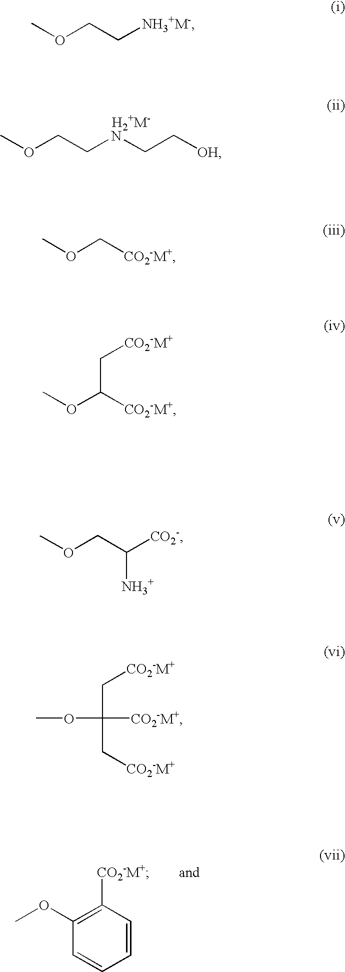
Y is —O—; and
Z is hydrogen or C₁₋₆alkyl;
or a pharmaceutically acceptable salt thereof.

12. A use according to claim 1, a composition according to claim 2, a product according to claim 3 or a method according to claim 4 wherein the NK-1 receptor antagonist is a compound of formula IV: 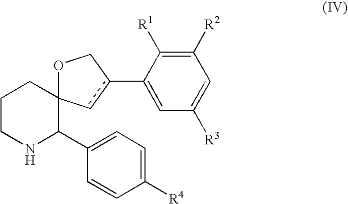
wherein
R¹ represents hydrogen, hydroxy, C₁₋₆alkyl, C₂₋₆alkenyl, C₃₋₇cycloalkyl, C₃₋₇cycloalkylC₁₋₄alkyl, C₁₋₆alkoxy, fluoroC₁₋₆alkoxy, C₁₋₆alkoxyC₁₋₄alkyl, C₁₋₆alkoxyC₁₋₄alkoxy, fluoroC₁₋₆alkoxyC₁₋₄alkyl, C₂₋₆alkenyloxy, C₃₋₇cycloalkoxy, C₃₋₇cycloalkylC₁₋₄alkoxy, phenoxy, benzyloxy, cyano, halogen, NR^{a}R^{b}, SR^{a}, SOR^{a}, SO₂R^{a}, OSO₂R^{a}, NR^{a}COR¹⁴, COR^{a}, CO₂R^{a}or CONR^{a}R^{b}where R^{a} and R^{b}each independently represent hydrogen, C₁₋₄alkyl or fluoroC₁₋₄alkyl;
R² represents hydrogen, halogen, C₁₋₆alkyl or C₁₋₆alkoxy;
or R¹ and R² may be joined together such that there is formed a 5- or 6-membered saturated or unsaturated ring containing one or two atoms selected from nitrogen, oxygen and sulphur, which ring is optionally substituted by a group selected from C₁₋₄alkyl, CF₃, ═O or ═S;
R³ represents hydrogen, halogen, C₁₋₆alkyl, fluoroC₁₋₆alkyl, C₁₋₆alkoxy, fluoroC₁₋₆alkoxy, C₃₋₇cycloalkyl, C₃₋₇cycloalkylC₁₋₄alkyl, cyano, SR^{a}, SOR^{a}, SO₂R^{a}, NR^{a}R^{b}, NR^{a}COR¹⁴, COR^{a}, CO₂R^{a}, CONR^{a}R^{b}or C₁₋₄alkyl substituted by cyano, CO₂R^{a}or CONR^{a}R^{b}where R^{a}and R^{b}are as previously defined;
R⁴ represents hydrogen, halogen, C₁₋₆alkyl, C₁₋₆alkoxy, CF₃, OCF₃, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy, where R^{a}and R^{b}are as previously defined; and
the broken line represents an optional double bond;
or a pharmaceutically acceptable salt thereof.

13. A use according to claim 1, a composition according to claim 2, a product according to claim 3 or a method according to claim 4 wherein the NK-1 receptor antagonist is a compound of formula V: 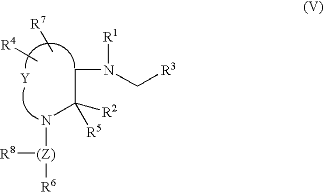
or a pharmaceutically acceptable salt thereof, wherein
Y is (CH₂)ₙ wherein n is an integer from 1 to 4, and wherein any one of the carbon-carbon single bonds in said (CH₂)ₙ may optionally be replaced by a carbon-carbon double bond, and wherein any one of the carbon atoms of said (CH₂)ₙ may optionally be substituted with R⁴, and wherein any one of the carbon atoms of said (CH₂)ₙ may optionally be substituted with R⁷;
Z is (CH₂)ₘ wherein m is an integer from 0 to 6, and wherein any one of the carbon-carbon single bonds of (CH₂)ₘ may optionally be replaced by a carbon-carbon double bond or a carbon-carbon triple bond, and any one of the carbon atoms of said (CH₂)ₘ may optionally be substituted with R⁸;
R¹ is hydrogen or C₁₋₈alkyl optionally substituted with hydroxy, C₁₋₄alkoxy or fluoro;
R² is a radical selected from hydrogen, C₁₋₆ straight or branched alkyl, C₃₋₇cycloalkyl wherein one of the CH₂ groups in said cycloalkyl may optionally be replaced by NH, oxygen or sulphur; aryl selected from phenyl and naphthyl; heteroaryl selected from indanyl, thienyl, furyl, pyridyl, thiazolyl, isothiazolyl, oxazolyl, isoxazolyl, triazolyl, tetrazolyl and quinolyl; phenyl-C₂₋₆alkyl, benzhydryl and benzyl, wherein each of said aryl and heteroaryl groups and the phenyl moieties of said benzyl, phenyl —C₂₋₆alkyl and benzhydryl may optionally be substituted with one or more substituents independently selected from halo, nitro, C₁₋₆ alkyl, C₁₋₆alkoxy, trifluoromethyl, amino, C₁₋₆alkylamino, C₁₋₆alkyl-O—CO, C₁₋₆alkyl-O—CO—C₁₋₆alkyl, C₁₋₆alkyl-CO—O, C₁₋₆alkyl-CO—C₁-C₆alkyl-O—, C₁₋₆alkyl-CO, C₁₋₆alkyl-CO—C₁₋₆alkyl-, di-C₁₋₆alkylamino, —CONH—C₁₋₆alkyl, C₁₋₆alkyl-CO—NH—C₁₋₆alkyl, —NHCOH and —NHCO—C₁alkyl; and wherein one of the phenyl moieties of said benzhydryl may optionally be replaced by naphthyl, thienyl, furyl or pyridyl;
R⁵ is hydrogen, phenyl or C₁₋₆alkyl;
or R² and R⁵ together with the carbon to which they are attached, form a saturated ring having from 3 to 7 carbon atoms wherein one of the CH₂ groups in said ring may optionally be replaced by oxygen, NH or sulfur;
R³ is aryl selected from phenyl and naphthyl; heteroaryl selected from indanyl, thienyl, furyl, pyridyl, thiazolyl, isothiazolyl, oxazolyl, isoxazolyl, triazolyl, tetrazolyl and quinolyl; and cycloalkyl having 3 to 7 carbon atoms wherein one of the (CH₂) groups in said cycloalkyl may optionally be replaced by NH, oxygen or sulphur;
wherein each of said aryl and heteroaryl groups may optionally be substituted with one or more substituents, and said C₃₋₇cycloalkyl may optionally be substituted with one or two substituents, each of said substituents being independently selected from halo, nitro, C₁₋₆alkyl, C₁₋₆alkoxy, trifluoromethyl, amino, C₁₋₆alkylamino, —CO—NH—C₁₋₆alkyl, C₁₋₆alkyl-CO—NH—C₁₋₆alkyl, —NHCOH and —NHCO—C₁₋₆alkyl;
R⁴ and R⁷ are each independently selected from hydroxy, halogen, halo, amino, oxo, cyano, methylene, hydroxymethyl, halomethyl, C₁₋₆alkylamino, di-C₁₋₆alkylamino, C₁₋₆alkoxy, C₁₋₆alkyl-O—CO, C₁₋₆alkyl-O—CO—C₁₋₆alkyl, C₁₋₆alkyl-CO—O, C₁₋₆alkyl-CO—C₁₋₆alkyl-O—, C₁₋₆alkyl-CO—, C₁₋₆alkyl-CO—C₁₋₆alkyl, and the radicals set forth in the definition of R²;
R⁶ is —NHCOR⁹, —NHCH₂R⁹, SO₂R⁸ or one of the radicals set forth in any of the definitions of R², R⁴ and R⁷;
R⁸ is oximino (═NOH) or one of the radicals set forth in any of the definitions of R², R⁴ and R⁷;
R⁹ is C₁₋₆alkyl, hydrogen, phenyl or phenylC₁₋₆alkyl;
with the proviso that (a) when m is 0, R⁸ is absent, (b) when R⁴, R⁶, R⁷ or R⁸ is as defined in R², it cannot form together with the carbon to which it is attached a ring with R⁵, and (c) when R⁴ and R⁷ are attached to the same carbon atom, then either each of R⁴ and R⁷ is independently selected from hydrogen, fluoro and C₁₋₆alkyl, or R⁴ and R⁷, together with the carbon to which they are attached, for a C₃₋₆ saturated carbocyclic ring that forms a spiro compound with the nitrogen-containing ring to which they are attached;
or a pharmaceutically acceptable salt thereof.

13. A use according to claim 1, a composition according to claim 2, a product according to claim 3 or a method according to claim 4 wherein the NK-1 receptor antagonist is a compound of formula VI: 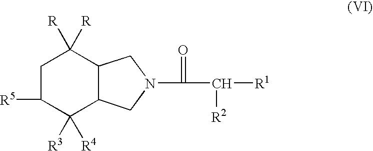
wherein:
radicals R are phenyl radicals optionally 2- or 3-substituted by a halogen atom or a methyl radical;
R¹ is optionally substituted phenyl, cyclohexadienyl, naphthyl, indenyl or optionally substituted heterocycle;
R² is H, halogen, OH, alkyl, aminoalkyl, alkylaminoalkyl, dialkylaminoalkyl, alkyloxy, alkylthio, acyloxy, carboxy, optionally substituted alkyloxycarbonyl, benzyloxycarbonyl, amino or acylamino;
R³ is optionally 2-substituted phenyl;
R⁴ is OH or fluorine when R⁵ is H;
or R⁴ and R⁵ are OH;
or R⁴ and R⁵ together form a bond;
or a pharmaceutically acceptable salt thereof.

14. A use according to claim 1, a composition according to claim 2, a product according to claim 3 or a method according to claim 4 wherein the NK-1 receptor antagonist is a compound of formula VII: 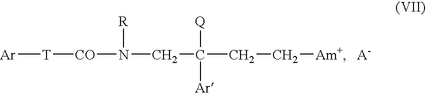
wherein:
Ar represents an optionally substituted mono-, di- or tricyclic aromatic or heteroaromatic group;
T represents a bond, a hydroxymethylene group, a C₁₋₄alkoxymethylene group or a C₁₋₅alkylene group;
Ar′ represents a phenyl group which is unsubstituted or substituted by one or more substituents selected from halogen, preferably chlorine or fluorine, trifluoromethyl, C₁₋₄alkoxy, C₁₋₄alkyl where the said substituents may be the same or different; a thienyl group; a benzothienyl group; a naphthyl group; or an indolyl group;
R represents hydrogen, C₁₋₄alkyl, ω-C₁₋₄alkoxyC₁₋₄alkyl, or ω-C₂₋₄alkanoyloxyC₂₋₄alkyl;
Q represents hydrogen;
or Q and R together form a 1,2-ethylene, 1,3-propylene or 1,4-butylene group;
Am⁺ represents the radical 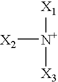
in which X₁, X₂ and X₃, together with the nitrogen atom to which they are attached, form an azabicyclic or azatricyclic ring system optionally substituted by a phenyl or benzyl group; and
A⁻ represents a pharmaceutically acceptable anion.

15. A use according to claim 1, a com position according to claim 2, a product according to claim 3 or a method according to claim 4 wherein the NK-1 receptor antagonist is a compound of formula VIII 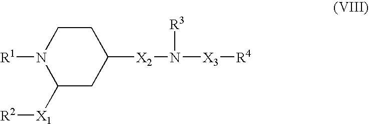
wherein:
R¹ represents an optionally substituted aralkyl, aryloxyalkyl, heteroaralkyl, aroyl, heteroaroyl, cycloalkylcarbonyl, aralkanoyl, heteroarylalkanoyl, aralkoxycarbonyl or arylcarbamoyl group or the acyl group of an α-amino acid optionally N-substituted by a lower alkanoyl or carbamoyl-lower alkanoyl group;
R² represents cycloalkyl or an optionally substituted aryl or heteroaryl group;
R³ represents hydrogen, alkyl, carbamoyl or an alkanoyl or alkenoyl group optionally substituted by carboxy or esterified or amidated carboxy;
R⁴ represents an optionally substituted aryl group or an optionally partially saturated heteroaryl group;
X₁ represents methylene, ethylene, a bond, an optionally ketalised carbonyl group or an optionally etherified hydroxymethylene group;
X₂ represents alkylene, carbonyl or a bond; and
X₃ represents carbonyl, oxo-lower alkyl, oxo(aza)-lower alkyl, or an alkyl group optionally substituted by phenyl, hydroxymethyl, optionally esterified or amidated carboxy, or (in other than the α-position) hydroxy;
or a pharmaceutically acceptable salt thereof.

16. A use according to claim 1, a composition according to claim 2, a product according to claim 3 or a method according to claim 4 wherein the NK-1 receptor antagonist is a compound of formula IX: 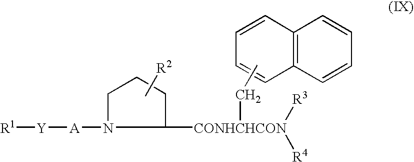
wherein:
R¹ is aryl, or a group of the formula: 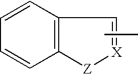
X is CH or N and
Z is O or N—R⁵, in which R⁵ is hydrogen or lower alkyl;
R² is hydroxy or lower alkoxy;
R³ is hydrogen or optionally substituted lower alkyl;
R⁴ is optionally substituted ar(lower)alkyl;
A is carbonyl or sulfonyl; and
Y is a bond or lower alkenylene;
or a pharmaceutically acceptable salt thereof.

17. A use according to claim 1, a composition according to claim 2, a product according to claim 3 or a method according to claim 4 wherein the NK-1 receptor antagonist is a compound of formula X: 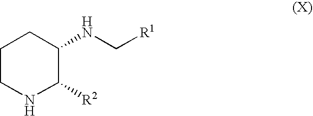
wherein:
R¹ is aryl selected from indanyl, phenyl and naphthyl; heteroaryl selected from thienyl, furyl, pyridyl and quinolyl; and cycloalkyl having 3 to 7 carbon atoms, wherein one of said carbon atoms may optionally be replaced by nitrogen, oxygen or sulfur; wherein each of said aryl and heteroaryl groups may optionally be substituted with one or more substituents, and said C₃₋₇cycloalkyl may optionally be substituted with one or two substituents, said substituents being independently selected from chloro, fluoro, bromo, iodo, nitro, C₁₋₁₀alkyl optionally substituted with from one to three fluoro groups, C₁₋₁₀alkoxy optionally substituted with from one to three fluoro groups, amino, C₁₋₁₀alkyl-S—, C₁₋₁₀alkyl-S(O)—, C₁₋₁₀alkyl-SO₂—, phenyl, phenoxy, C₁₋₁₀alkyl-SO₂NH—, C₁₋₁₀alkyl-SO₂NH—C₁₋₁₀akyl-, C₁₋₁₀alkylamino-diC₁₋₁₀alkyl-, cyano, hydroxy, cycloalkoxy having 3 to 7 carbon atoms, C₁₋₆alkylamino, C₁₋₆dialkylamino, HC(O)NH— and C₁₋₁₀alkyl-C(O)NH—; and
R² is thienyl, benzhydryl, naphthyl or phenyl optionally substituted with from one to three substituents independently selected from chloro, bromo, fluoro, iodo, cycloalkoxy having 3 to 7 carbon atoms, C₁₋₁₀alkyl optionally substituted with from one to three fluoro groups and C₁₋₁₀alkoxy optionally substituted with from one to three fluoro groups;
or a pharmaceutically acceptable salt thereof.

18. A use according to claim 1, a composition according to claim 2, a product according to claim 3 or a method according to claim 4 wherein the NK-1 receptor antagonist is a compound of formula XI: 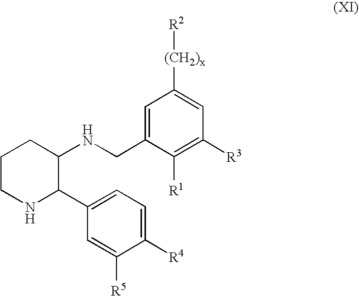
wherein:
R¹ is a C₁₋₄alkoxy group;
R² is 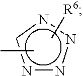
R³ is a hydrogen or halogen atom;
R⁴ and R⁵ may each independently represent a hydrogen or halogen atom, or a C₁₋₄alkyl, C₁₋₄alkoxy or trifluoromethyl group;
R⁶ is a hydrogen atom, a C₁₋₄alkyl, (CH₂)ₘcyclopropyl, —S(O)ₙC₁₋₄alkyl, phenyl, NR⁷R⁸, CH₂C(O)CF₃ or trifluoromethyl group;
R⁷ and R⁸ may each independently represent a hydrogen atom, or a C₁₋₄alkyl or acyl group;
x represents zero or 1;
n represents zero, 1 or 2; and
m represents zero or 1;
or a pharmaceutically acceptable salt thereof.

19. A use according to claim 1, a composition according to claim 2, a product according to claim 3 or a method according to claim 4 wherein the NK-1 receptor antagonist is a compound of formula XII: 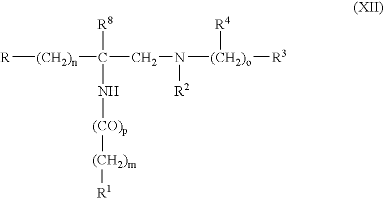
wherein:
m is zero, 1, 2 or 3;
n is zero or 1;
o is zero, 1 or 2;
p is zero or 1;
R is phenyl, 2- or 3-indolyl, 2- or 3-indolinyl, benzothienyl, benzofuranyl, or naphthyl;
which R groups may be substituted with one or two halo, C₁₋₃alkoxy, trifluoromethyl, C₁₋₄alkyl, phenyl-C₁₋₃alkoxy, or C₁₋₄alkanoyl groups;
R¹ is trityl, phenyl, diphenylmethyl, phenoxy, phenylthio, piperazinyl, piperidinyl, pyrrolidinyl, morpholinyl, indolinyl, indolyl, benzothienyl, hexamethyleneiminyl, benzofuranyl, tetrahydropyridinyl, quinolinyl, isoquinolinyl, reduced quinolinyl, reduced isoquinolinyl, phenyl-(C₁₋₄alkyl)-, phenyl-(C₁₋₄alkoxy)-, quinolinyl-(C₁₋₄alkyl)-, isoquinolinyl-(C₁₋₄alkyl)-, reduced quniolinyl-(C₁₋₄alkyl)-, reduced isoquinolinyl-(C₁₋₄alkyl)-, benzoyl-(C₁₋₁₃alkyl)-, C₁₋₄alkyl, or —NH—CH₂—R⁵;
any one of which R¹ groups may be substituted with halo, C₁₋₄alkyl, C₁₋₄alkoxy, trifluoromethyl, amino, C₁₋₄alkylamino, di(C₁₋₄alkyl)amino, or C₂₋₄alkanoylamino;
or any one of which R¹ groups may be substituted with phenyl, piperazinyl, C₃₋₈cycloalkyl, benzyl, C₁₋₄alkyl, piperidinyl, pyridinyl, pyrimidinyl, C₂₋₆alkanoylamino, pyrrolidinyl, C₂₋₆alkanoyl, or C₁₋₄alkoxycarbonyl;
any one of which groups may be substituted with halo, C₁₋₄alkyl, C₁₋₄alkoxy, trifluoromethyl, amino, C₁₋₄alkylamino, di(C₁₋₄alkyl)amino, or C₂₋₄alkanoylamino;
or R¹ is amino, a leaving group, hydrogen, C₁₋₄alkylamino, or di(C₁₋₄alkyl)amino;
R⁵ is pyridyl, anilino-(C₁₋₃alkyl)-, or anilinocarbonyl;
R² is hydrogen, C₁₋₄alkyl, C₁₋₄alkylsulfonyl, carboxy-(C₁₋₃alkyl)-, C₁₋₃alkoxycarbonyl-(C₁₋₃alkyl)-, or —CO—R⁶;
R⁶ is hydrogen, C₁₋₄alkyl, C₁₋₃haloalkyl, phenyl, C₁₋₃alkoxy, C₁₋₃hydroxyalkyl, amino, C₁₋₄alkylamino, di(C₁₋₄alkyl)amino, or —(CH₂)_{q}—R⁷;
q is zero to 3;
R⁷ is carboxy, C₁₋₄alkoxycarbonyl, C₁₋₄alkylcarbonyloxy, amino, C₁₋₄alkylamino, di(C₁₋₄alkyl)amino, C₁₋₆alkoxycarbonylamino, or phenoxy, phenylthio, piperazinyl, piperidinyl, pyrrolidinyl, morpholinyl, indolinyl, indolyl, benzothienyl, benzofuranyl, quinolinyl, phenyl-(C₁₋₄alkyl)-, quinolinyl-(C₁₋₄alkyl)-, isoquinolinyl-(C₁₋₄alkyl)-, reduced quinolinyl-(C₁₋₄alkyl)-, reduced isoquinolinyl-(C₁₋₄alkyl)-, benzoyl-C₁₋₃alkyl;
any one of which aryl or heterocyclic R⁷ groups may be substituted with halo, trifluoromethyl, C₁₋₄alkoxy, C₁₋₄alkyl, amino, C₁₋₄alkylamino, di(C₁₋₄alkyl)amino, or C₂₋₄alkanoylamino;
or any one of which R⁷ groups may be substituted with phenyl, piperazinyl, C₃₋₆cycloalkyl, benzyl, piperidinyl, pyridinyl, pyrimidinyl, pyrrolidinyl, C₂₋₆alkanoyl, or C₁₋₄alkoxycarbonyl;
any of which groups may be substituted with halo, trifluoromethyl, amino, C₁₋₄alkoxy, C₁₋₄alkyl, C₁₋₄alkylamino, di(C₁₋₄alkyl)amino, or C₂₋₄alkanoylamino;
R⁸ is hydrogen or C₁₋₆alkyl;
R³ is phenyl, phenyl-(C₁₋₆alkyl)-, C₃₋₈cycloalkyl, C₅₋₈cycloalkenyl, C₁₋₈alkyl, naphthyl, C₂₋₈alkenyl, or hydrogen;
any one or which groups except hydrogen may be substituted with one or two halo, C₁₋₃alkoxy, C₁₋₃alkylthio, nitro, trifluoromethyl, or C₁₋₁₃alkyl groups; and
R⁴ is hydrogen or C₁₋₃alkyl;
with the proviso that if R¹ is hydrogen or halo, R³ is phenyl, phenyl —(C₆alkyl)-, C₃₋₈cycloalkyl, C₅₋₈cycloalkenyl, or naphthyl;
or a pharmaceutically acceptable salt thereof.

20. A use, composition, product or method according to any one of the preceding claims wherein the NK-1 receptor antagonist is orally active, long acting and CNS-penetrant.

21. A use according to Claim 1, a composition according to claim 2, a product according to claim 3 or a method according to claim 4 wherein the NK-1 receptor antagonist is selected from the classes of compounds described in EP-A-0577394, WO-A-9508549, WO-A-9518124, WO-A-9523798 or WO-A-9605181.

22. A use according to claim 1, a composition according to claim 2, a product according to claim 3 or a method according to claim 4 wherein the NK-1 receptor antagonist is selected from
4-(3-(1,2,4-triazolo)methyl)-2(S)-(3,5-bis(trifluoromethyl)benzyloxy)-3(S)-phenyl-morpholine;
4-(3-(1,2,4-triazolo)methyl)-2(S)-(3,5-bis(trifluoromethyl)benzyloxy)-3(R)-phenyl-morpholine;
4-(3-(5-oxo-1H,4H-1,2,4-triazolo)methyl)-2(S)-(3,5-bis(trifluoromethyl)benzyloxy)-3(S)-phenyl-morpholine;
2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine;
2-(R)-(1-(R)-(3,5-bis(trifluoromethyl)phenyl)ethoxy)-4-(5-(dimethylamino) methyl-1,2,3-triazol-4-yl)methyl-3-(S)-phenylmorpholine;
2-(R)-(1-(R)-(3,5-bis(trifluoromethyl)phenyl)ethoxy)-4-(5-(dimethylamino) methyl-1,2,3-triazol-4-yl)methyl-3-(S)-(4-fluorophenyl)morpholine;
2-(R)-(1-(S)-(3,5-bis(trifluoromethyl)phenyl)-2-hydroxyethoxy)-3-(S)-(4-fluorophenyl)-4-(1,2,4-triazol-3-yl)methylmorpholine;
2-(S)-(3,5-bis(trifluoromethyl)benzyloxy)-3-(S)-phenyl-4-(3-(5-oxo-1H,4H-1,2,4-triazolo)methyl)morpholine N-oxide;
2-(S)-(3,5-bis(trifluoromethyl)benzyloxy)-3-(S)-phenyl-4-(3-(4-(ethoxycarbonyloxy-1-ethyl)-5-oxo-1H-1,2,4-triazolo)methyl)morpholine;
2-(R)-(1-(R)-(3,5-bis(trifluoromethyl)phenyl)ethoxy)-3-(S)-(4-fluorophenyl)-4-(3-(4-monophosphoryl-5-oxo-1H-1,2,4-triazolo)methyl)morpholine;
2-(R)-(1-(R)-(3,5-bis(trifluoromethyl)phenyl)ethoxy)-3-(S)-(4-fluorophenyl)-4-(3-(1-monophosphoryl-5-oxo-1H-1,2,4-triazolo)methyl)morpholine;
2-(R)-(1-(R)-(3,5-bis(trifluoromethyl)phenyl)ethoxy)-3-(S)-(4-fluorophenyl)-4-(3-(2-monophosphoryl-5-oxo-1H-1,2,4-triazolo)methyl)morpholine;
2-(R)-(1-(R)-(3,5-bis(trifluoromethyl)phenyl)ethoxy)-3-(S)-(4-fluorophenyl)-4-(3-(5-oxyphosphoryl-1H-1,2,4-triazolo)methyl)morpholine;
2-(S)-(1-(R)-(3,5-bis(trifluoromethyl)phenyl)ethoxy)-3-(S)-(4-fluorophenyl)-4-(3-(1-monophosphoryl-5-oxo-4H-1,2,4-triazolo)methyl)morpholine;
(3R,5R,6S)-3-(2-methoxy-5-(trifluoromethoxy)phenyl)-6-phenyl-1-oxa-7-aza-spiro[4.5]decane;
(3R,5R,6S)-3-(2-methoxy-5-(trifluoromethyl)phenyl)-6-phenyl-1-oxa-7-aza-spiro[4.5]decane;
(3R,5R,6S)-7-benzyl-3-[2-methoxy-5-(trifluoromethoxy)phenyl]-6-phenyl-1-oxa-7-aza-spiro[4.5]decane;
(3R,5R,6S)-3-(2-methoxy-5-trifluoromethoxyphenyl)-6-phenyl-1-oxa-7-aza-spiro[4.5]decane;
(3R,5R,6S)-3,6-bis(phenyl)-1-oxa-7-aza-spiro[4.5]decane;
(3R,5R,6S)-7-benzyl-3-(2-methoxy-5-trifluoromethoxyphenyl)-6-phenyl-1-oxa-7-aza-spiro[4.5]decane;
(±)(3R*,5R*,6S*)-3-(2-methoxyphenyl)-6-phenyl-1-oxa-7-(phenylmethoxycarbonyl)aza-spiro [4.5]decane;
(3R,5R,6S)-3-(2-methoxyphenyl)-6-phenyl-1-oxa-7-aza-spiro [4.5]decane;
(3S,5R,6S)-3-(2-cyclopropoxy-5-(trifluoromethoxy)phenyl)-6-phenyl-1-oxa-7-aza-spiro[4.5]decane;
(3R,5R,6S)-3-[2-cyclopropoxy-5-(trifluoromethoxy)phenyl]-6-phenyl-1-oxa-7-aza-spiro[4.5]decane;
(3S,5R,6S)-3-[2-cyclopropoxy-5-(trifluoromethyl)phenyl]-6-phenyl-1-oxa-7-aza-spiro[4.5]decane;
(2S,3S)-cis-3-(2-methoxybenzylamino)-2-phenylpiperidine;
(3aS,4S,7aS)-7,7-diphenyl-4-(2-methoxyphenyl)-2-[(2S)-(2-methoxyphenyl)propionyl]perhydroisoindol-4-ol;
(+) 1-[2-[3-(3,4-dichlorophenyl)-1-[(3-isopropoxyphenyl)acetyl]-3-piperidinyl]ethyl]-4-phenyl-1-azabicyclo[2,2,2]octane;
(2R*,4S*)-2-benzyl-1-(3,5-dimethylbenzoyl)-N-(4-quinolinylmethyl)-4-piperidineamine; 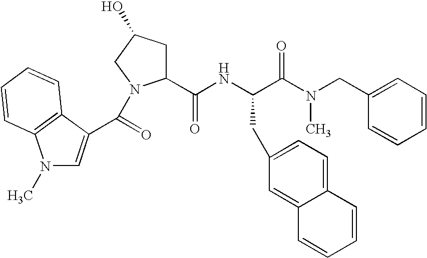
(2S,3S)-3-(2-methoxy-5-trifluoromethoxybenzyl)-amino-2-phenylpiperidine;
(2-methoxy-5-tetrazol-1-yl-benzyl)-([2S,3S]-2-phenyl-piperidin-3-yl)-amine;
2-methoxy-5-(5-trifluoromethyl-tetrazol-1-yl)-benzyl-([2S,3S]-2-phenyl-piperidin-3-yl)-amine; and
[N-(2-methoxybenzyl)acetylamino]-3-(1H-indol-3-yl)-2-[N-(2-(4-piperidin-1-yl)piperidin-1-yl)acetylamino]propane;
or a pharmaceutically acceptable salt thereof.

23. A process for preparing a pharmaceutical composition comprising combining a COX-2 inhibitor and a NK-1 receptor antagonist with a pharmaceutically acceptable carrier.

24. The use of a NK-1 receptor antagonist for the manufacture of a medicament for the combined use with a cyclooxygenase-2 inhibitor for preventing or reducing the risk of developing an inflammatory disorder, for halting or slowing the progression of an inflammatory disorder, or for preventing or reducing the risk of occurrence or recurrence of an inflammatory disorder.

25. The use of a cyclooxygenase-2 inhibitor for the preparation of a medicament for the combined use with a NK-1 receptor antagonist for preventing or reducing the risk of developing an inflammatory disorder, for halting or slowing the progression of an inflammatory disorder, or for preventing or reducing the risk of occurrence or recurrence of an inflammatory disorder.

26. A use, composition, product or method according to any one of the preceding claims wherein the inflammatory disorder is selected from rheumatoid arthritis, degenerative joint diseases, osteoarthritis, bursitis, tendinitis, ankylosing spondylitis, gout and synovitis.
